(19) United States
(12) Patent Application Publication
(10) Pub. No.: US 2008/0044399 A1
(43) Pub. Date: Feb. 21, 2008

(54) Vitamin E supplementation for reducing cardiovascular events in individuals with DM and the Hp 2-2 genotype

(76) Inventor: Andrew Levy, Haifa (IL)

Correspondence Address: PEARL COHEN ZEDEK LATZER, LLP, 1500 BROADWAY 12TH FLOOR, NEW YORK, NY, 10036, US

(21) Appl. No.: 11/798,488

(22) Filed: May 14, 2007

Related U.S. Application Data
(63) Continuation-in-part of application No. 10/645,530, filed on Aug. 22, 2003, now abandoned.
Application No. 10/645,530 is a continuation of application No. 09/815,016, filed on Mar. 23, 2001, now Pat. No. 6,613,519.
Application No. 09/815,016 is a continuation-in-part of application No. 09/556,469, filed on Apr. 20, 2000, now Pat. No. 6,251,608.
(60) Provisional application No. 60/273,538, filed on Mar. 7, 2001.

(51) Int. Cl. A61K 38/44; A61P 9/00; C12Q 1/68

(52) U.S. Cl. 424/94.4; 435/6

(57) ABSTRACT

This invention relates to methods and compositions of determining the benefit of therapy using vitamin-E for the treatment of cardiovascular events in individuals with diabetes melitus based on their Haptoglobin phenotype and the treatment of the cardiovascular events using vitamin-E based on the haptoglobin phenotype.

### CROSS-REFERENCE TO RELATED APPLICATIONS

This is a continuation-in-part of U.S. patent application Ser. No. 10/645,530, filed Aug. 22, 2003, which is a continuation of U.S. patent application Ser. No. 09/815,016, filed Mar. 23, 2001, now U.S. Pat. No. 6,613,519, issued Sep. 2, 2003, which is a continuation-in-part of U.S. patent application Ser. No. 09/556,469, filed Apr. 20, 2000, now U.S. Pat. No. 6,251,608, issued Jun. 26, 2001, and which also claims the benefit of priority from U.S. Provisional Patent Application No. 60/273,538, filed Mar. 7, 2001. This Application also claims the benefit of priority from U.S. Provisional Patent Application No. 60/437,439, filed Jan. 2, 2003, all of which are incorporated herein by reference in their entirety.

### FIELD OF INVENTION

This invention is directed to methods and compositions of determining the benefit of therapy using vitamin-E for the treatment of cardiovascular events in individuals with diabetes melitus based on their Haptoglobin phenotype and the treatment of the cardiovascular events using vitamin-E based on the haptoglobin phenotype.

### BACKGROUND OF THE INVENTION

Extensive preclinical and observational studies showing apparent benefit from vitamin E in preventing cardiovascular events created an atmosphere in which over 40% of cardiologists were routinely prescribing high dose vitamin E. Over the past 10 years, several prospective randomized clinical trials have investigated whether vitamin E supplementation provides cardiovascular protection. The overwhelming consensus from these studies is that vitamin E supplementation does not provide cardiovascular benefit. To the contrary, meta-analysis of these studies suggests high dose vitamin E supplementation may increase mortality and several opinion articles have called for a moratorium on prescription of high dose vitamin E supplements.

High dose antioxidant therapy may only provide benefit to individuals who suffer from particularly high levels of oxidative stress.

Accordingly, there is a need to develop criteria based on which treatment with vitamin-E may be safely prescribed and likewise, optimal dosage determined for both vitamin-E either alone, or with other compounds capable of reducing oxidative stress.

### SUMMARY OF THE INVENTION

In one embodiment, the invention provides a method of determining prognosis for a diabetic subject having a cardiovascular complication, to benefit from supplementation of vitamin-E, comprising the step of obtaining a biological sample from the subject; and determining the subject's haptoglobin allelic genotype, whereby a subject expressing the Hp-2-2 genotype will benefit from supplementation of vitamin-E

In another embodiment, the invention provides a method of treating, or inhibiting or suppressing or reducing symptoms associated with a diabetic subject having a cardiovascular complication, comprising the step of contacting the subject with an effective amount of a composition comprising glutathione peroxidase or its isomer, metabolite, and/or salt therefore, and vitamin-E, thereby treating vascular complication.

In one embodiment, the invention provides a composition for treating a cardiovascular complication in a subject comprising: a therapeutically effective amount of a composition comprising glutathione peroxidase or its isomer, metabolite, and/or salt therefore and vitamin-E.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will be better understood from a reading of the following detailed description taken in conjunction with the drawings in which like reference designators are used to designate like elements, and in which:

FIG. 1. shows Participants flow chart;

FIG. 2 shows Kaplan Meier plot of the composite endpoint in vitamin E and placebo treated Hp 2-2 DM individuals. Events are CV death, myocardial infarction or stroke. There were a total of 18 patients (3.8%) who had events in the placebo group and 5 patients who had events in the vitamin E group (1.0%). There was a significant decrease in the composite endpoint in the vitamin E group compared to the placebo group (HR 0.26 (95% CI 0.13-0.69), p=0.004 by Log-Rank analysis); and

FIG. 3 shows Kaplan Meier plot of the composite endpoint according to Hp genotype in the registry. Events are cardiovascular death, myocardial infarction or stroke. There were 285 Hp 1-1, 1248 Hp 2-1 and 527 Hp 2-2 individuals in the registry with 6 individuals who had an event (2.1%) in the Hp 1-1 group, 24 individuals who had an event (1.9%) in the Hp 2-1 group, and 22 individuals who had an event (4.2%) in the Hp 2-2 group (p=0.005 by Log-Rank analysis for the difference in the event rate between Hp 2-2 and non-Hp 2-2).

### DETAILED DESCRIPTION OF THE INVENTION

This invention relates in one embodiment to methods and in another embodiment, to compositions for determining the benefit of therapy using vitamin-E for the treatment of cardiovascular events in individuals with diabetes melitus based on their Haptoglobin phenotype and the treatment of the cardiovascular events using vitamin-E based on the haptoglobin phenotype

In one embodiment, the haptoglobin (Hp) genotype helps to identify patients with high levels of oxidative stress and who will benefit from antioxidant therapy with vitamin E. The Hp gene is polymorphic with two common classes of alleles denoted 1 and 2. It was demonstrated that the Hp 2 allele protein product is an inferior antioxidant compared to the Hp 1 allele protein product. These differences in antioxidant protection are profoundly accentuated in the diabetic state resulting in a marked relative increase in oxidative stress in Hp 2 transgenic mice and Hp 2-2 individuals with DM.

The distribution of the three Hp genotypes in western societies is approximately 16% Hp 1-1, 36% Hp 2-2 and 48% Hp 2-1. In another embodiment, an interaction between the Hp genotype and DM was demonstrated to have an effect on the development of cardiovascular events. In certain embodiments, Hp 2-2 DM individuals have been shown to have as much as a 500% increase in cardiovascular events as compared to Hp 1-1 and Hp 2-1 DM individuals. In one embodiment, vitamin E provides cardiovascular benefit to DM individuals with the Hp 2-2 genotype.

Accordingly and in one embodiment, provided herein is a method of determining prognosis for a diabetic subject having a cardiovascular complication, to benefit from supplementation of vitamin-E, comprising the step of obtaining a biological sample from the subject; and determining the subject's haptoglobin allelic genotype, whereby a subject expressing the Hp-2-2 genotype will benefit from supplementation of vitamin-E.

In one embodiment, vitamin E is added to foods in one of its more chemically stable forms, e.g., .alpha.-tocopherol acetate (also known as .alpha.-tocopheryl acetate). Four different forms of vitamin E (the alcohol and ester forms of synthetic racemic (rac) vitamin E and the alcohol and ester forms of natural (RRR) vitamin E) are commercially available, and because of their differences in bioactivities and molecular weights, are assigned different values of specific activity (IU per milligram) according to the National Formulary as follows: 1 mg all-rac-.alpha.-tocopherol acetate=1.00 IU 1 mg all-rac-.alpha-tocopherol=1.10 IU 1 mg RRR—.alpha-tocopherol acetate=1.36 IU 1 mg RRR-.alpha-tocopherol=1.49 IU.

In one embodiment, the vitamin E is selected from the group consisting of alpha, beta, gamma and delta tocopherols, alpha, beta, gamma and delta tocotrienols, and combinations thereof. In another embodiment, the alpha tocopherol group is selected from the group consisting of synthetic (all-rac) and natural (RRR) alpha-tocopherols, alpha-tocopheryl acetates, and alpha-tocopheryl succinates.

In one embodiment, the Hp 2-2 genotype is associated with a markedly increased incidence of cardiovascular disease in the setting of DM. In another embodiment, Hp 2-2 Apo E^{-/-} mice have increased atherosclerotic plaque macrophage content, iron and oxidation as compared to Hp 1-1 Apo E^{-/-} mice. In one embodiment, reverse cholesterol transport is impaired in Hp 2-2 DM mice.

In another embodiment, the antioxidant function of Hp is due to its ability to neutralize hemoglobin which is capable of generating the highly reactive hydroxyl radical. Micro-hemorrhages resulting in liberation of extravascular extracorpuscular hemoglobin are of increased frequency and severity in diabetic atherosclerosis. The Hp 1-1 protein is superior to the Hp 2-2 protein in protecting against extracorpuscular hemoglobin as a result of its better ability to prevent release of heme from the Hp-hemoglobin complex and to promote uptake of the Hp-hemoglobin complex via the macrophage CD163 receptor.

Haptoglobin is inherited by two co-dominant autosomal alleles situated on chromosome 16 in humans, these are Hp1 and Hp2. There are three phenotypes Hp1-1, Hp2-1 and Hp2-2. Haptoglobin molecule is a tetramer comprising of four polypeptide chains, two alpha and two beta chains, of which alpha chain is responsible for polymorphism because it exists in two forms, alpha-1 and alpha-2. Hp1-1 is a combination of two alpha-1 chains along with two beta chains. Hp2-1 is a combination of one α-1 chain and one alpha-2 chain along with two beta chains. Hp2-2 is a combination of two α-2 chains and two beta chains. Hp1-1 individuals have greater hemoglobin binding capacity when compared to those individuals with Hp2-1 and Hp2-2. The gene differentiation to Hp-2 from Hp-1 resulted in a dramatic change in the biophysical and biochemical properties of the haptoglobin protein encoded by each of the 2 alleles. The gene differentiation to Hp-2 from Hp-1 resulted in a dramatic change in the biophysical and biochemical properties of the haptoglobin protein encoded by each of the 2 alleles. The haptoglobin phenotype of any individual, 1-1, 2-1 or 2-2, is readily determined in one embodiment, from 10 μl of plasma by gel electrophoresis.

In one embodiment, antioxidant therapy may be beneficial in specific subgroups with increased oxidative stress. Oxidative Stress refers in one embodiment to a loss of redox homeostasis (imbalance) with an excess of reactive oxidative species (ROS) by the singular process of oxidation. Both redox and oxidative stress are associated in another embodiment, with an impairment of antioxidant defensive capacity as well as an overproduction of ROS. In another embodiment, the methods and compositions of the invention are used in the treatment of complications or pathologies resulting from oxidative stress in subjects.

In one embodiment, activated neutrophils and tissue macrophages use an NADPH cytochrome b-dependent oxidase for the reduction of molecular oxygen to superoxide anions. In another embodiment, fibroblasts, are also be stimulated to produce ROS in response to pro-inflammatory cytokines. In another embodiment, prolonged production of high levels of ROS cause severe tissue damage. In one embodiment, high levels of ROS cause DNA mutations that can lead to neoplastic transformation. Therefore and in one embodiment, cells in injured tissues such as glial cells and neurons, must be able to protect themselves against the toxic effects of ROS. In one embodiment ROS-detoxifying enzymes have an important role in epithelial wound repair. In another embodiment, the glutathione peroxidase mimetics provided in the compositions and compounds provided herein, replace the ROS detoxifying enzymes described herein.

In one embodiment, overproduction of reactive oxygen species (ROS) including hydrogen peroxide (H₂O₂), superoxide anion (O.₂⁻); nitric oxide (NO.) and singlet oxygen (¹O₂) creates an oxidative stress, resulting in the amplification of the inflammatory response. Self-propagating lipid peroxidation (LPO) against membrane lipids begins and endothelial dysfunction ensues. Endogenous free radical scavenging enzymes (FRSEs) such as superoxide dismutase (SOD), glutathione peroxidase (GPX) and catalase are, involved in the disposal of O.₂⁻ and H₂O₂. First, SOD catalyses the dismutation of O.₂⁻ to H₂O₂ and molecular oxygen (O.₂), resulting in selective O.₂⁻ scavenging. Then, GPX and catalase independently decompose H₂O.₂ to H₂O. In another embodiment, ROS is released from the active neutrophils in the inflammatory tissue, attacking DNA and/or membrane lipids and causing chemical damage, including in one embodiment, to healthy tissue. When free radicals are generated in excess or when FRSEs are defective, H₂O₂ is reduced into hydroxyl radical (OH.), which is one of the highly reactive ROS responsible in one embodiment for initiation of lipid peroxidation of cellular membranes. In another embodiment, organic peroxide-induced lipid peroxidation is implicated as one of the essential mechanisms of toxicity in the death of hippocampal neurons. In one embodiment, an indicator of the oxidative stress in the cell is the level of lipid peroxidation and its final product is MDA. In another embodiment the level of lipid peroxidation increases in inflammatory diseases, such as meningitis in one embodiment. In one embodiment, the compounds provided herein and in another embodiment, are represented by the compounds of formula I-X, are effective antioxidants, capable of reducing lipid peroxidation, or in another embodiment, are effective as anti-inflammatory agents.

In another embodiment, the methods and systems provided herein of determining prognosis for a diabetic subject having a cardiovascular complication, to benefit from supplementation of vitamin-E, comprising the step of obtaining a biological sample from the subject; and determining the subject's haptoglobin allelic genotype, whereby a subject expressing the Hp-2-2 genotype will benefit from supplementation of vitamin-E, is effected by a signal amplification method, whereby said signal amplification method is PCR, LCR (LAR), Self-Sustained Synthetic Reaction (3SR/NASBA), Q-Beta (Qβ) Replicase reaction, or a combination thereof.

In another embodiment, the signal amplification methods provided herein, which in another embodiment, can be carried out using the systems provided herein, may amplify a DNA molecule or an RNA molecule. In another embodiment, signal amplification methods used as part of the present invention include, but are not limited to PCR, LCR (LAR), Self-Sustained Synthetic Reaction (3SR/NASBA) or a Q-Beta (Q.beta.) Replicase reaction.

Polymerase Chain Reaction (PCR): The polymerase chain reaction (PCR), refers in one embodiment to a method of increasing the concentration of a segment of target sequence in a mixture of genomic DNA without cloning or purification. This technology provides one approach to the problems of low target sequence concentration. PCR can be used to directly increase the concentration of the target to an easily detectable level. This process for amplifying the target sequence involves the introduction of a molar excess of two oligonucleotide primers which are complementary to their respective strands of the double-stranded target sequence to the DNA mixture containing the desired target sequence. The mixture is denatured and then allowed to hybridize. Following hybridization, the primers are extended with polymerase so as to form complementary strands. The steps of denaturation, hybridization (annealing), and polymerase extension (elongation) can be repeated as often as needed, in order to obtain relatively high concentrations of a segment of the desired target sequence.

The length of the segment of the desired target sequence is determined by the relative positions of the primers with respect to each other, and, therefore, this length is a controllable parameter. Because the desired segments of the target sequence become the dominant sequences (in terms of concentration) in the mixture, in one embodiment, they are said to be “PCR-amplified.”

Ligase Chain Reaction (LCR or LAR): The ligase chain reaction [LCR; referred to, in another embodiment as “Ligase Amplification Reaction” (LAR)] has developed into a well-recognized alternative method of amplifying nucleic acids. In LCR, four oligonucleotides, two adjacent oligonucleotides which uniquely hybridize to one strand of target DNA, and a complementary set of adjacent oligonucleotides, which hybridize to the opposite strand are mixed in one embodiment and DNA ligase is added to the mixture. Provided that there is complete complementarity at the junction, ligase will covalently link each set of hybridized molecules. In another embodiment of LCR, two probes are ligated together only when they base-pair with sequences in the target sample, without gaps or mismatches. Repeated cycles of denaturation, and ligation amplify a short segment of DNA. LCR has is used in combination with PCR in one embodiment, to achieve enhanced detection of single-base changes. In another embodiment, because the four oligonucleotides used in this assay can pair to form two short ligatable fragments, there is the potential for the generation of target-independent background signal. The use of LCR for mutant screening is limited in another embodiment, to the examination of specific nucleic acid positions.

Self-Sustained Synthetic Reaction (3SR1NASBA): The self-sustained sequence replication reaction (3SR) refers in one embodiment, to a transcription-based in vitro amplification system that can exponentially amplify RNA sequences at a uniform temperature. The amplified RNA is utilized in certain embodiments, for mutation detection. In an embodiment of this method, an oligonucleotide primer is used to add a phage RNA polymerase promoter to the 5′ end of the sequence of interest. In a cocktail of enzymes and substrates that includes a second primer, reverse transcriptase, RNase H, RNA polymerase and ribo- and deoxyribonucleoside triphosphates, the target sequence undergoes repeated rounds of transcription, cDNA synthesis and second-strand synthesis to amplify the area of interest. The use of 3SR to detect mutations is kinetically limited to screening small segments of DNA (e.g., 200-300 base pairs).

Q-Beta (Qβ.) Replicase: In one embodiment of the method, a probe which recognizes the sequence of interest is attached to the replicatable RNA template for Qβ. replicase. A previously identified major problem with false positives resulting from the replication of unhybridized probes has been addressed through use of a sequence-specific ligation step. However, available thermostable DNA ligases are not effective on this RNA substrate, so the ligation must be performed by T4 DNA ligase at low temperatures (37° C.). This prevents the use of high temperature as a means of achieving specificity as in the LCR, the ligation event can be used to detect a mutation at the junction site, but not elsewhere.

The basis of the amplification procedure in the PCR and LCR is the fact that the products of one cycle become usable templates in all subsequent cycles, consequently doubling the population with each cycle. The final yield of any such doubling system can be expressed as: (1+X)ⁿ=y, where “X” is the mean efficiency (percent copied in each cycle), “n” is the number of cycles, and “y” is the overall efficiency, or yield of the reaction (Mullis, PCR Methods Applic., 1:1, 1991). If every copy of a target DNA is utilized as a template in every cycle of a polymerase chain reaction, then the mean efficiency is 100%. If 20 cycles of PCR are performed, then the yield will be 2²⁰, or 1,048,576 copies of the starting material. If the reaction conditions reduce the mean efficiency to 85%, then the yield in those 20 cycles will be only 1.85²⁰, or 220,513 copies of the starting material. In other words, a PCR running at 85% efficiency will yield only 21% as much final product, compared to a reaction running at 100% efficiency. A reaction that is reduced to 50% mean efficiency will yield less than 1% of the possible product.

In practice, routine polymerase chain reactions rarely achieve the theoretical maximum yield, and PCRs are usually run for more than 20 cycles to compensate for the lower yield. At 50% mean efficiency, it would take 34 cycles to achieve the million-fold amplification theoretically possible in 20, and at lower efficiencies, the number of cycles required becomes prohibitive. In addition, any background products that amplify with a better mean efficiency than the intended target will become the dominant products.

In another embodiment, many variables can influence the mean efficiency of PCR, including target DNA length and secondary structure, primer length and design, primer and dNTP concentrations, and buffer composition, to name but a few. Contamination of the reaction with exogenous DNA (e.g., DNA spilled onto lab surfaces) or cross-contamination is also a major consideration. Reaction conditions must be carefully optimized for each different primer pair and target sequence, and the process can take days, even for an experienced investigator. The laboriousness of this process, including numerous technical considerations and other factors, presents a significant drawback to using PCR in the clinical setting. Indeed, PCR has yet to penetrate the clinical market in a significant way. The same concerns arise with LCR, as LCR must also be optimized to use different oligonucleotide sequences for each target sequence. In addition, both methods require expensive equipment, capable of precise temperature cycling.

Many applications of nucleic acid detection technologies, such as in studies of allelic variation, involve not only detection of a specific sequence in a complex background, but also the discrimination between sequences with few, or single, nucleotide differences. One method of the detection of allele-specific variants by PCR is based upon the fact that it is difficult for Taq polymerase to synthesize a DNA strand when there is a mismatch between the template strand and the 3′ end of the primer. An allele-specific variant may be detected by the use of a primer that is perfectly matched with only one of the possible alleles; the mismatch to the other allele acts to prevent the extension of the primer, thereby preventing the amplification of that sequence. This method has a substantial limitation in that the base composition of the mismatch influences the ability to prevent extension across the mismatch, and certain mismatches do not prevent extension or have only a minimal effect.

A similar 3′-mismatch strategy is used with greater effect to prevent ligation in the LCR. Any mismatch effectively blocks the action of the thermostable ligase, but LCR still has the drawback of target-independent background ligation products initiating the amplification. Moreover, the combination of PCR with subsequent LCR to identify the nucleotides at individual positions is also a clearly cumbersome proposition for the clinical laboratory.

In another embodiment, the methods and systems provided herein for providing a prognosis for a diabetic subject to benefit from supplementation of vitamin-E, comprising the steps of: obtaining a biological sample from a subject; determining the Haptoglobin (Hp) genotype in the biological sample that is effected by a direct detection method such as a cycling probe reaction (CPR), or a branched DNA analysis, or a combination thereof in other embodiments.

The direct detection method according to one embodiment is a cycling probe reaction (CPR) or a branched DNA analysis. When a sufficient amount of a nucleic acid to be detected is available, there are advantages to detecting that sequence directly, instead of making more copies of that target, (e.g., as in PCR and LCR). Most notably, a method that does not amplify the signal exponentially is more amenable to quantitative analysis. Even if the signal is enhanced by attaching multiple dyes to a single oligonucleotide, the correlation between the final signal intensity and amount of target is direct. Such a system has an additional advantage that the products of the reaction will not themselves promote further reaction, so contamination of lab surfaces by the products is not as much of a concern. Traditional methods of direct detection including Northern and Southern band RNase protection assays usually require the use of radioactivity and are not amenable to automation. Recently devised techniques have sought to eliminate the use of radioactivity and/or improve the sensitivity in automatable formats. Two examples are the “Cycling Probe Reaction” (CPR), and “Branched DNA” (bDNA).

Cycling probe reaction (CPR): The cycling probe reaction (CPR) (Duck et al., BioTech., 9:142, 1990), uses a long chimeric oligonucleotide in which a central portion is made of RNA while the two termini are made of DNA. Hybridization of the probe to a target DNA and exposure to a thermostable RNase H causes the RNA portion to be digested. This destabilizes the remaining DNA portions of the duplex, releasing the remainder of the probe from the target DNA and allowing another probe molecule to repeat the process. The signal, in the form of cleaved probe molecules, accumulates at a linear rate. While the repeating process increases the signal, the RNA portion of the oligonucleotide is vulnerable to RNases that may carried through sample preparation.

In another embodiment, the methods and systems provided herein of determining prognosis for a diabetic subject having a cardiovascular complication, to benefit from supplementation of vitamin-E, comprising the step of obtaining a biological sample from the subject; and determining the subject's haptoglobin allelic genotype, whereby a subject expressing the Hp-2-2 genotype will benefit from supplementation of vitamin-E, is effected by at least one sequence change, which employs in one embodiment a restriction fragment length polymorphism (RFLP analysis), or an allele specific oligonucleotide (ASO) analysis, a Denaturing/Temperature Gradient Gel Electrophoresis (DGGE/TGGE), a Single-Strand Conformation Polymorphism (SSCP) analysis or a Dideoxy fingerprinting (ddF) or their combination in other embodiments.

Restriction fragment length polymorphism (RFLP): For detection of single-base differences between like sequences, the requirements of the analysis are often at the highest level of resolution. For cases in which the position of the nucleotide in question is known in advance, several methods have been developed for examining single base changes without direct sequencing. For example, if a mutation of interest happens to fall within a restriction recognition sequence, a change in the pattern of digestion can be used as a diagnostic tool (e.g., restriction fragment length polymorphism [RFLP] analysis).

Single point mutations have been also detected by the creation or destruction of RFLPs. Mutations are detected and localized by the presence and size of the RNA fragments generated by cleavage at the mismatches. Single nucleotide mismatches in DNA heteroduplexes are also recognized and cleaved by some chemicals, providing an alternative strategy to detect single base substitutions, generically named the “Mismatch Chemical Cleavage” (MCC) (Gogos et al., Nucl. Acids Res., 18:6807-6817, 1990). However, this method requires the use of osmium tetroxide and piperidine, two highly noxious chemicals which are not suited for use in a clinical laboratory.

RFLP analysis suffers from low sensitivity and requires a large amount of sample. When RFLP analysis is used for the detection of point mutations, it is, by its nature, limited to the detection of only those single base changes which fall within a restriction sequence of a known restriction endonuclease. Moreover, the majority of the available enzymes have 4 to 6 base-pair recognition sequences, and cleave too frequently for many large-scale DNA manipulations (Eckstein and Lilley (eds.), Nucleic Acids and Molecular Biology, vol. 2, Springer-Verlag, Heidelberg, 1988). Thus, it is applicable only in a small fraction of cases, as most mutations do not fall within such sites.

A handful of rare-cutting restriction enzymes with 8 base-pair specificities have been isolated and these are widely used in genetic mapping, but these enzymes are few in number, are limited to the recognition of G+C-rich sequences, and cleave at sites that tend to be highly clustered (Barlow and Lehrach, Trends Genet., 3:167, 1987). Recently, endonucleases encoded by group I introns have been discovered that might have greater than 12 base-pair specificity (Perhnan and Butow, Science 246:1106, 1989), but again, these are few in number.

Allele specific oligonucleotide (ASO): allele-specific oligonucleotides (ASOs), can be designed to hybridize in proximity to the mutated nucleotide, such that a primer extension or ligation event can bused as the indicator of a match or a mis-match. Hybridization with radioactively labeled allelic specific oligonucleotides (ASO) also has been applied to the detection of specific point mutations (Conner et al., Proc. Natl. Acad. Sci., 80:278-282, 1983). The method is based on the differences in the melting temperature of short DNA fragments differing by a single nucleotide. Stringent hybridization and washing conditions can differentiate between mutant and wild-type alleles. The ASO approach applied to PCR products also has been extensively utilized by various researchers to detect and characterize point mutations in ras genes (Vogelstein et al., N. Eng. J. Med., 319:525-532, 1988; and Farr et al., Proc. Natl. Acad. Sci., 85:1629-1633, 1988), and gsp/gip oncogenes (Lyons et al., Science 249:655-659, 1990). Because of the presence of various nucleotide changes in multiple positions, the ASO method requires the use of many oligonucleotides to cover all possible oncogenic mutations.

Denaturing/Temperature Gradient Gel Electrophoresis (DGGE/TGGE): Two other methods rely on detecting changes in electrophoretic mobility in response to minor sequence changes. One of these methods, termed “Denaturing Gradient Gel Electrophoresis” (DGGE) is based on the observation that slightly different sequences will display different patterns of local melting when electrophoretically resolved on a gradient gel. In this manner, variants can be distinguished, as differences in melting properties of homoduplexes versus heteroduplexes differing in a single nucleotide can detect the presence of mutations in the target sequences because of the corresponding changes in their electrophoretic mobilities. The fragments to be analyzed, usually PCR products, are “clamped” at one end by a long stretch of G-C base pairs (30-80) to allow complete denaturation of the sequence of interest without complete dissociation of the strands. The attachment of a GC “clamp” to the DNA fragments increases the fraction of mutations that can be recognized by DGGE (Abrams et al., Genomics 7:463-475, 1990). Attaching a GC clamp to one primer is critical to ensure that the amplified sequence has a low dissociation temperature (Sheffield et al., Proc. Natl. Acad. Sci., 86:232-236, 1989; and Lerman and Silverstein, Meth. Enzymol., 155:482-501, 1987). Modifications of the technique have been developed, using temperature gradients (Wartell et al., Nucl. Acids Res., 18:2699-2701, 1990), and the method can be also applied to RNA:RNA duplexes (Smith et al., Genomics 3:217-223, 1988).

Limitations on the utility of DGGE include the requirement that the denaturing conditions must be optimized for each type of DNA to be tested. Furthermore, the method requires specialized equipment to prepare the gels and maintain the needed high temperatures during electrophoresis. The expense associated with the synthesis of the clamping tail on one oligonucleotide for each sequence to be tested is also a major consideration. In addition, long running times are required for DGGE. The long running time of DGGE was shortened in a modification of DGGE called constant denaturant gel electrophoresis (CDGE) (Borrensen et al., Proc. Natl. Acad. Sci. USA 88:8405, 1991). CDGE requires that gels be performed under different denaturant conditions in order to reach high efficiency for the detection of mutations.

A technique analogous to DGGE, termed temperature gradient gel electrophoresis (TGGE), uses a thermal gradient rather than a chemical denaturant gradient (Scholz, et al., Hum. Mol. Genet. 2:2155, 1993). TGGE requires the use of specialized equipment which can generate a temperature gradient perpendicularly oriented relative to the electrical field. TGGE can detect mutations in relatively small fragments of DNA therefore scanning of large gene segments requires the use of multiple PCR products prior to running the gel.

Single-Strand Conformation Polymorphism (SSCP): Another common method, called “Single-Strand Conformation Polymorphism” (SSCP) was developed by Hayashi, Sekya and colleagues (reviewed by Hayashi, PCR Meth. Appl., 1:34-38, 1991) and is based on the observation that single strands of nucleic acid can take on characteristic conformations in non-denaturing conditions, and these conformations influence electrophoretic mobility. The complementary strands assume sufficiently different structures that one strand may be resolved from the other. Changes in sequences within the fragment will also change the conformation, consequently altering the mobility and allowing this to be used as an assay for sequence variations (Orita, et al., Genomics 5:874-879, 1989).

The SSCP process involves denaturing a DNA segment (e.g., a PCR product) that is labeled on both strands, followed by slow electrophoretic separation on a non-denaturing polyacrylamide gel, so that intra-molecular interactions can form and not be disturbed during the run. This technique is extremely sensitive to variations in gel composition and temperature. A serious limitation of this method is the relative difficulty encountered in comparing data generated in different laboratories, under apparently similar conditions.

Dideoxy fingerprinting (ddF): The dideoxy fingerprinting (ddF) is another technique developed to scan genes for the presence of mutations (Liu and Sommer, PCR Methods Appli., 4:97, 1994). The ddF technique combines components of Sanger dideoxy sequencing with SSCP. A dideoxy sequencing reaction is performed using one dideoxy terminator and then the reaction products are electrophoresed on nondenaturing polyacrylamide gels to detect alterations in mobility of the termination segments as in SSCP analysis. While ddF is an improvement over SSCP in terms of increased sensitivity, ddF requires the use of expensive dideoxynucleotides and this technique is still limited to the analysis of fragments of the size suitable for SSCP (i.e., fragments of 200-300 bases for optimal detection of mutations).

In addition to the above limitations, all of these methods are limited as to the size of the nucleic acid fragment that can be analyzed. For the direct sequencing approach, sequences of greater than 600 base pairs require cloning, with the consequent delays and expense of either deletion sub-cloning or primer walking, in order to cover the entire fragment. SSCP and DGGE have even more severe size limitations. Because of reduced sensitivity to sequence changes, these methods are not considered suitable for larger fragments. Although SSCP is reportedly able to detect 90% of single-base substitutions within a 200 base-pair fragment, the detection drops to less than 50% for 400 base pair fragments. Similarly, the sensitivity of DGGE decreases as the length of the fragment reaches 500 base-pairs. The ddF technique, as a combination of direct sequencing and SSCP, is also limited by the relatively small size of the DNA that can be screened.

Determination of a haptoglobin phenotype may, as if further exemplified in the Examples section that hereinbelow, may be accomplished directly in one embodiment, by analyzing the protein gene products of the haptoglobin gene, or portions thereof. Such a direct analysis is often accomplished using an immunological detection method. In one embodiment, the methods and systems provided herein for providing a prognosis for development of a diabetic subject to benefit from supplementation of vitamin-E, comprising the steps of: obtaining a biological sample from a subject; determining the Haptoglobin (Hp) genotype in the biological sample by an immunological detection method, such as is a radio-immunoassay (RIA) in one embodiment, or an enzyme linked immunosorbent assay (ELISA), a western blot, an immunohistochemical analysis, or fluorescence activated cell sorting (FACS), or a combination thereof in other embodiments.

Immunological detection methods are fully explained in, for example, “Using Antibodies: A Laboratory Manual” (Ed Harlow, David Lane eds., Cold Spring Harbor Laboratory Press (1999)) and those familiar with the art will be capable of implementing the various techniques summarized hereinbelow as part of the present invention. All of the immunological techniques require antibodies specific to at least one of the two haptoglobin alleles. Immunological detection methods suited for use as part of the present invention include, but are not limited to, radio-immunoassay (RIA), enzyme linked immunosorbent assay (ELISA), western blot, immunohistochemical analysis, and fluorescence activated cell sorting (FACS).

Radio-immunoassay (RIA): In one version, this method involves precipitation of the desired substrate, haptoglobin in this case and in the methods detailed hereinbelow, with a specific antibody and radiolabelled antibody binding protein (e.g., protein A labeled with I.sup.125) immobilized on a perceptible carrier such as agarose beads. The number of counts in the precipitated pellet is proportional to the amount of substrate. In an alternate version of the RIA, A labeled substrate and an unlabelled antibody binding protein are employed. A sample containing an unknown amount of substrate is added in varying amounts. The decrease in precipitated counts from the labeled substrate is proportional to the amount of substrate in the added sample.

Enzyme linked immunosorbent assay (ELISA): This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabelled or enzyme linked as described hereinabove. Detection may be by autoradiography, colorimetric reaction or chemiluminescence. This method allows both quantitation of an amount of substrate and determination of its identity by a relative position on the membrane which is indicative of a migration distance in the acrylamide gel during electrophoresis.

Immunohistochemical analysis: This method involves detection of a substrate in situ in fixed cells by substrate specific antibodies. The substrate specific antibodies may be enzyme linked or linked to fluorophores. Detection is by microscopy and subjective evaluation. If enzyme linked antibodies are employed, a calorimetric reaction may be required.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

It will be appreciated by one ordinarily skilled in the art that determining the haptoglobin phenotype of an individual, either directly or genetically, may be effected using any suitable biological sample derived from the examined individual, including, but not limited to, blood, plasma, blood cells, saliva or cells derived by mouth wash, and body secretions such as urine and tears, and from biopsies, etc.

In one embodiment, the effectiveness of the compounds provided herein derive from special structural features of the heterocyclic compounds provided herein. In one embodiment, having a large number of electrons in the π orbital overlap around the transition metal incorporated allows the formation of π-bonds and the donation of an electron to terminate free radicals formed by ROS. In one embodiment, the glutathione peroxidase mimetic used in the method of inhibiting or suppressing free radical formation, causing in another embodiment, lipid peroxidation and inflammation, is the product of formula (I):

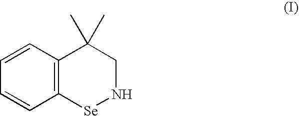

where nitrogen has 4 electrons in the p-orbital, thereby making 2 electrons available for π bonds; and each carbon has 2 electron in the p-orbital thereby making 1 electron available for π bonds; and selenium has 6 electrons in the p-orbital, thereby making 3 electrons available for π bonds, for a total of 7 electrons, since in another embodiment, the adjacent benzene ring removes two carbons from participating in the π-bond surrounding the metal. Upon a loss of electron by the transition metal, following termination of free radicals, the number of electrons in the π-bond overlap, is reduced to 6 π electron, a very stable aromatic sextet. In vitro and in vivo studies with the compound of formula 1, a show in one embodiment, that glutahion peroxidase or its isomer, metabolite, and/or salt therefore is capable of protecting cells against reactive oxygen species.

Four types of GPx have been identified: cellular GPx (cGPx), gastrointestinal GPx, extracellular GPx, and phospholipid hydroperoxide GPx. cGPx, also termed in one embodiment, GPX1, is ubiquitously distributed. It reduces hydrogen peroxide as well as a wide range of organic peroxides derived from unsaturated fatty acids, nucleic acids, and other important biomolecules. At peroxide concentrations encountered under physiological conditions and in another embodiment, it is more active than catalase (which has a higher Kₘ for hydrogen peroxide) and is active against organic peroxides in another embodiment. Thus, cGPx represents a major cellular defense against toxic oxidant species.

Peroxides, including hydrogen peroxide (H₂O₂), are one of the main reactive oxygen species (ROS) leading to oxidative stress. H₂O₂ is continuously generated by several enzymes (including superoxide dismutase, glucose oxidase, and monoamine oxidase) and must be degraded to prevent oxidative damage. The cytotoxic effect of H₂O₂ is thought to be caused by hydroxyl radicals generated from iron-catalyzed reactions, causing subsequent damage to DNA, proteins, and membrane lipids.

In one embodiment, administration of GPx or its pharmaceutically acceptable salt, its functional derivative, its synthetic analog or a combination thereof, is used in the methods and compositions of the invention.

In one embodiment, the glutathione peroxidase, is represented by formula I:

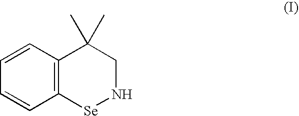

In one embodiment, the compound of formula (II), refers to benzisoselen-azoline or -azine derivatives represented by the following general formula: 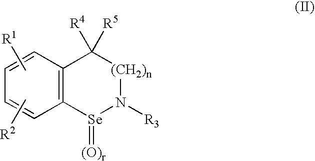
where: R¹, R²=hydrogen; lower alkyl; OR⁶; —(CH₂)ₘ NR⁶R⁷; —(CH₂)_{q}NH₂; —(CH₂)ₘNHSO₂(CH₂)₂ NH₂; —NO₂; —CN; —SO₃H; —N⁺(R⁵)₂O⁻; F; Cl; Br; I; —(CH₂)ₘR⁸; —(CH₂)ₘCOR⁸; —S(O)NR⁶R⁷; —SO₂NR⁶R⁷; —CO(CH₂)ₚCOR⁸; R⁹; R³=hydrogen; lower alkyl; aralkyl; substituted aralkyl; —(CH₂)ₘCOR⁸; —(CH₂)_{q}R⁸; —CO(CH₂)ₚCOR⁸; —(CH₂)ₘSO₂R⁸; —(CH₂)ₘS(O)R⁸; R⁴=lower alkyl; aralkyl; substituted aralkyl; —(CH₂)ₚCOR⁸; —(CH₂)ₚR⁸; F; R⁵=lower alkyl; aralkyl; substituted aralkyl; R⁶=lower alkyl; aralkyl; substituted aralkyl; —(CH₂)ₙCOR⁸; —(CH₂)_{q}R⁸; R⁷=lower alkyl; aralkyl; substituted aralkyl; —(CH₂)ₘCOR⁸; R⁸=lower alkyl; aralkyl; substituted aralkyl; aryl; substituted aryl; heteroaryl; substituted heteroaryl; hydroxy; lower alkoxy; R⁹; R⁹=

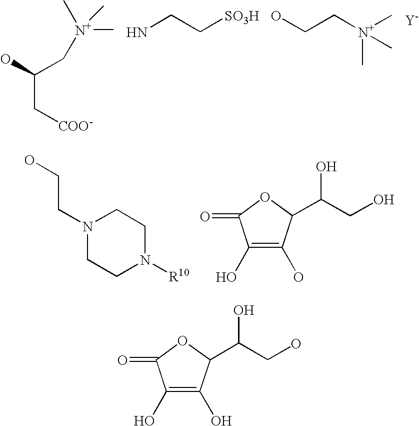

R¹⁰=hydrogen; lower alkyl; aralkyl or substituted aralkyl; aryl or substituted aryl; Y⁻ represents the anion of a pharmaceutically acceptable acid; n=0, 1; m=0, 1, 2; p=1, 2, 3; q=2, 3, 4 and r=0, 1.

In one embodiment, “Alkyl” refers to monovalent alkyl groups preferably having from 1 to about 12 carbon atoms, more preferably 1 to 8 carbon atoms and still more preferably 1 to 6 carbon atoms. This term is exemplified by groups such as methyl, ethyl, n-propyl, isopropyl, n-butyl, isobutyl, tert-butyl, n-hexyl, n-octyl, tert-octyl and the like. The term “lower alkyl” refers to alkyl groups having 1 to 6 carbon atoms.

In another embodiment, “Aralkyl” refers to -alkylene-aryl groups preferably having from 1 to 10 carbon atoms in the alkylene moiety and from 6 to 14 carbon atoms in the aryl moiety. Such alkaryl groups are exemplified by benzyl, phenethyl, and the like.

“Aryl” refers in another embodiment, to an unsaturated aromatic carbocyclic group of from 6 to 14 carbon atoms having a single ring (e.g., phenyl).or multiple condensed rings (e.g., naphthyl or anthryl). Preferred aryls include phenyl, naphthyl and the like. Unless otherwise constrained by the definition for the individual substituent, such aryl groups can optionally be substituted with from 1 to 3 substituents selected from the group consisting of alkyl, substituted alkyl, alkoxy, alkenyl, alkynyl, amino, aminoacyl, aminocarbonyl, alkoxycarbonyl, aryl, carboxyl, cyano, halo, hydroxy, nitro, trihalomethyl and the like.

It will be appreciated that aryl and heteroaryl groups (including bicyclic aryl groups) can be unsubstituted or substituted, wherein substitution includes replacement of one or more of the hydrogen atoms thereon independently with any one or more of the following moieties including, but not limited to: aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —C(O)N(Rₓ)₂; —OC(O)Rₓ; —OCO₂Rₓ; —OC(O)N(Rₓ)₂; —N(Rₓ)₂; —ORₓ; —SRₓ; —S(O)Rₓ; —S(O)2Rₓ; —NRₓ(CO)Rₓ; —N(Rₓ)CO₂Rₓ; —N(Rₓ)S(O)₂Rₓ; —N(Rₓ)C(O)N(Rₓ)₂; —S(O)₂N(Rₓ)₂; wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl, heteroaryl, -(alkyl)aryl or -(alkyl)heteroaryl substituents described above and herein may be substituted or unsubstituted. Additionally, it will be appreciated, that any two adjacent groups taken together may represent a 4, 5, 6, or 7-membered substituted or unsubstituted alicyclic or heterocyclic moiety.

In one embodiment, the glutathione peroxidase or its isomer, metabolite, and/or salt therefore, used in the methods and compositions provided herein is an organoselenium compound. The term “organoselenium” refers in one embodiment to organic compound comprising at least one selenium atom. Preferred classes of organoselenium glutathione peroxidase mimetics include benzisoselenazolones, diaryl diselenides and diaryl selenides. In one embodiment, provided herein are compositions and methods of treating cardiovascular complication in a diabetic subject, comprising organoselenium compounds, thereby increasing endogenous anti-oxidant ability of the cells, or in another embodiment, scavenging free radicals causing apoptosis of cardiovascular organs and tissues and their associated pathologies.

Accordingly and in one embodiment, provided herein is a composition for treating a cardiovascular complication in a subject comprising: a therapeutically effective amount of a composition comprising glutathione peroxidase or its isomer, metabolite, and/or salt therefore and vitamin-E.

In another embodiment, the glutathione peroxidase or its isomer, metabolite, and/or salt therefore used in the compositions and methods provided herein, is represented by the compound of formula III: 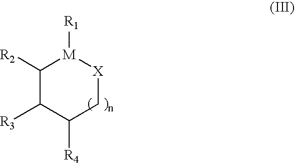
wherein,
the compound of formula I is a ring; and
   X is O or NH
   M is Se or Te
   n is 0-2
   R₁ is oxygen; and
   forms an oxo complex with M; or
   R₁ is oxygen or NH; and
forms together with the metal, a 4-7 member ring, which optionally is substituted by an oxo group; or
forms together with the metal, a first 4-7 member ring, which is optionally substituted by an oxo group, wherein said first ring is fused with a second 4-7 member ring, wherein said second 4-7 member ring is optionally substituted by alkyl, alkoxy, nitro, aryl, cyano, amino, halogen, or —NH(C═O)R or —SO₂R where R is alkyl or aryl;
R₂, R₃ and R₄ are independently hydrogen, alkyl, oxo, amino or together with the organometallic ring to which two of the substituents are attached, a fused 4-7 member ring system wherein said 4-7 member ring is optionally substituted by alkyl, alkoxy, nitro, aryl, cyano, amino, halogen, or —NH(C═O)R or —SO₂R where R is alkyl or aryl; wherein R₄ is not an alkyl; and
wherein if R₂, R₃ and R₄ are hydrogen and R₁ forms an oxo complex with M, n is 0 then M is Te; or
if R₂, R₃ and R₄ are hydrogen and R₁ is an oxygen that forms together with the metal an unsubstituted, saturated, 5 member ring, n is 0 then M is Te; or
if R₁ is an oxo group, and n is 0, R₂ and R₃ form together with the organometallic ring a fused benzene ring, R₄ is hydrogen, then M is Se; or
   if R₄ is an oxo group, and R₂ and R₃ form together with the organometallic ring a fused benzene ring, R₁ is oxygen, n is 0 and forms together with the metal a first 5 member ring, substituted by an oxo group α to R₁, and said ring is fused to a second benzene ring, then M is Te.

In one embodiment, a 4-7 member ring group refers to a saturated cyclic ring. In another embodiment the 4-7 member ring group refers to an unsaturated cyclic ring. In another embodiment the 4-7 member ring group refers to a heterocyclic unsaturated cyclic ring. In another embodiment the 4-7 member ring group refers to a heterocyclic saturated cyclic ring. In one embodiment the 4-7 member ring is unsubstituted. In one embodiment, the ring is substituted by one or more of the following: alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl.

In one embodiment, substituent groups may be attached via single or double bonds, as appropriate, as will be appreciated by one skilled in the art.

According to embodiments herein, the term alkyl as used throughout the specification and claims may include both “unsubstituted alkyls” and/or “substituted alkyls”, the latter of which may refer to alkyl moieties having substituents replacing hydrogen on one or more carbons of the hydrocarbon backbone. In another embodiment, such substituents may include, for example, a halogen, a hydroxyl, an alkoxyl, a silyloxy, a carbonyl, and ester, a phosphoryl, an amine, an amide, an imine, a thiol, a thioether, a thioester, a sulfonyl, an amino, a nitro, or an organometallic moiety. It will be understood by those skilled in the art that the moieties substituted on the hydrocarbon chain may themselves be substituted, if appropriate. For instance, the substituents of a substituted alkyl may include substituted and unsubstituted forms of amines, imines, amides, phosphoryls (including phosphonates and phosphines), sulfonyls (including sulfates and sulfonates), and silyl groups, as well as ethers, thioethers, selenoethers, carbonyls (including ketones, aldehydes, carboxylates, and esters), —CF₃, and —CN. Of course other substituents may be applied. In another embodiment, cycloalkyls may be further substituted with alkyls, alkenyls, alkoxys, thioalkyls, aminoalkyls, carbonyl-substituted alkyls, CF₃, and CN. Of course other substituents may be applied.

In another embodiment, a compound of formula IV is provided, wherein M, R₁ and R₄ are as described above for formula III.

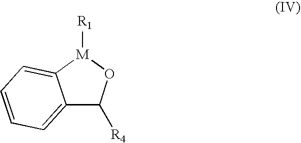

In another embodiment, a compound of formula V is provided, wherein M, R₂, R₃ and R₄ are as described above for formula III.

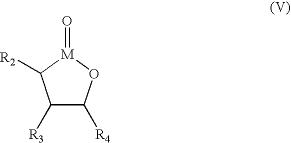

In another embodiment, a compound of formula VI is provided, wherein M, R₂, R₃ and R₄ are as described above for formula III.

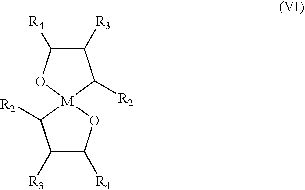

In another embodiment, a compound of formula (VII) is provided, wherein M, R₂ and R₃ are as described above for formula III

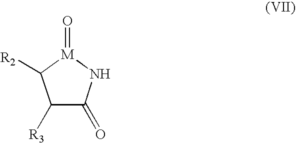

In another embodiment, a compound of formula VIII is provided, wherein M, R₂ and R₃ are as described above for formula III.

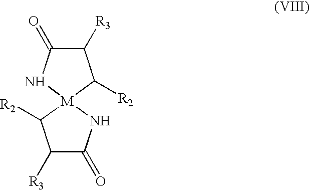

In one embodiment, the compound of formula III, used in the compositions and methods provided herein, is represented by any one of the following compounds or their combinations:

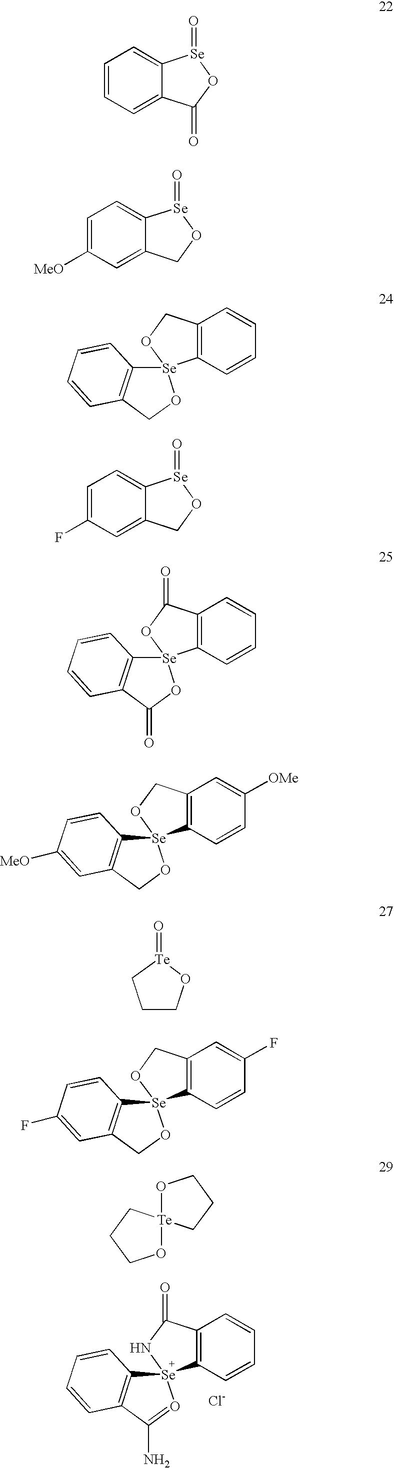

In another embodiment, the glutathione peroxidase or its isomer, metabolite, and/or salt therefore used in the compositions and methods provided herein, is represented by the compound of formula IX: 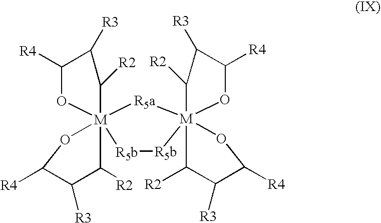
wherein,

M is Se or Te;

R₂, R₃ or R₄ are independently hydrogen, alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; or R₂, R₃ or R₄ together with the organometallic ring to which two of the substituents are attached, is a fused 4-7 membered ring system, wherein said 4-7 membered ring is optionally substituted by alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; and

R₅ₐ or R_{5b} is one or more oxygen, carbon, or nitrogen atoms and forms a neutral complex with the chalcogen.

In one embodiment, the compound represented formula (IX), is represented by the compound of formula X:

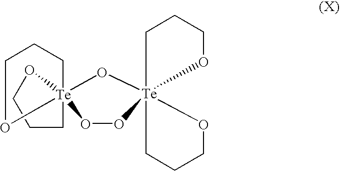

In one embodiment, the compounds represented by formula I-X, mimic the in-vivo activity of glutathione peroxidase. The term “mimic” refers, in one embodiment to comparable, identical, or superior activity, in the context of conversion, timing, stability or overall performance of the compound, or any combination thereof.

Biologically active derivatives or analogs of the proteins described herein include in one embodiment peptide mimetics. These mimetics can be based, for example, on the protein's specific amino acid sequence and maintain the relative position in space of the corresponding amino acid sequence. These peptide mimetics possess biological activity similar to the biological activity of the corresponding peptide compound, but possess a “biological advantage” over the corresponding amino acid sequence with respect to, in one embodiment, the following properties: solubility, stability and susceptibility to hydrolysis and proteolysis.

Methods for preparing peptide mimetics include modifying the N-terminal amino group, the C-terminal carboxyl group, and/or changing one or more of the amino linkages in the peptide to a non-amino linkage. Two or more such modifications can be coupled in one peptide mimetic molecule. Other forms of the proteins and polypeptides described herein and encompassed by the claimed invention, include in another embodiment, those which are “functionally equivalent.” In one embodiment, this term, refers to any nucleic acid sequence and its encoded amino acid which mimics the biological activity of the protein, or polypeptide or functional domains thereof in other embodiments.

In one embodiment, the composition further comprises a carrier, excipient, lubricant, flow aid, processing aid or diluent, wherein said carrier, excipient, lubricant, flow aid, processing aid or diluent is a gum, starch, a sugar, a cellulosic material, an acrylate, calcium carbonate, magnesium oxide, talc, lactose monohydrate, magnesium stearate, colloidal silicone dioxide or mixtures thereof.

In another embodiment, the composition further comprises a binder, a disintegrant, a buffer, a protease inhibitor, a surfactant, a solubilizing agent, a plasticizer, an emulsifier, a stabilizing agent, a viscosity increasing agent, a sweetener, a film forming agent, or any combination thereof.

In one embodiment, the compositions provided herein are used for the treatment of a cardiovascular condition in a diabetic subject, may be present in the form of suspension or dispersion form in solvents or fats, in the form of a nonionic vesicle dispersion or else in the form of an emulsion, preferably an oil-in-water emulsion, such as a cream or milk, or in the form of an ointment, gel, cream gel, sun oil, solid stick, powder, aerosol, foam or spray.

In one embodiment, the composition is a particulate composition coated with a polymer (e.g., poloxamers or poloxamines). Other embodiments of the compositions of the invention incorporate particulate forms protective coatings, protease inhibitors or permeation enhancers for various routes of administration, including parenteral, pulmonary, nasal and oral. In one embodiment the pharmaceutical composition is administered parenterally, paracancerally, transmucosally, transdermally, intramuscularly, intravenously, intradermally, subcutaneously, intraperitonealy, intraventricularly, or intracranially.

In some embodiments, the compositions and methods provided herein permit direct application to the site where it is needed. In the practice of the methods provided herein, it is contemplated that virtually any of the compositions provided herein can be employed.

In one embodiment, the compositions of this invention may be in the form of a pellet, a tablet, a capsule, a solution, a suspension, a dispersion, an emulsion, an elixir, a gel, an ointment, a cream, or a suppository.

In another embodiment, the composition is in a form suitable for oral, intravenous, intraarterial, intramuscular, subcutaneous, parenteral, transmucosal, transdermal, or topical administration. In one embodiment the composition is a controlled release composition. In another embodiment, the composition is an immediate release composition. In one embodiment, the composition is a liquid dosage form. In another embodiment, the composition is a solid dosage form.

In another embodiment, the compositions provided herein are suitable for oral, intraoral, rectal, parenteral, topical, epicutaneous, transdermal, subcutaneous, intramuscular, intranasal, sublingual, buccal, intradural, intraocular, intrarespiratory, nasal inhalation or a combination thereof. In one embodiment, the step of administering the compositions provided herein, in the methods provided herein is carried out as oral administration, or in another embodiment, the administration of the compositions provided herein is intraoral, or in another embodiment, the administration of the compositions provided herein is rectal, or in another embodiment, the administration of the compositions provided herein is parenteral, or in another embodiment, the administration of the compositions provided herein is topical, or in another embodiment, the administration of the compositions provided herein is epicutaneous, or in another embodiment, the administration of the compositions provided herein is transdermal, or in another embodiment, the administration of the compositions provided herein is subcutaneous, or in another embodiment, the administration of the compositions provided herein is intramuscular, or in another embodiment, the administration of the compositions provided herein is intranasal, or in another embodiment, the administration of the compositions provided herein is sublingual, or in another embodiment, the administration of the compositions provided herein is buccal, or in another embodiment, the administration of the compositions provided herein is intradural, or in another embodiment, the administration of the compositions provided herein is intraocular, or in another embodiment, the administration of the compositions provided herein is intrarespiratory, or in another embodiment, the administration of the compositions provided herein is nasal inhalation or in another embodiment, the administration of the compositions provided herein is a combination thereof.

The compounds utilized in the methods and compositions of the present invention may be present in the form of free bases in one embodiment or pharmaceutically acceptable acid addition salts thereof in another embodiment. In one embodiment, the term “pharmaceutically-acceptable salts” embraces salts commonly used to form alkali metal salts and to form addition salts of free acids or free bases. The nature of the salt is not critical, provided that it is pharmaceutically-acceptable. Suitable pharmaceutically-acceptable acid addition salts of compounds of Formula I are prepared in another embodiment, from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, example of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, b-hydroxybutyric, salicylic, galactaric and galacturonic acid. Suitable pharmaceutically-acceptable base addition salts include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine (N-methylglucamine) and procaine. All of these salts may be prepared by conventional means from the corresponding compound by reacting, in another embodiment, the appropriate acid or base with the compound.

In one embodiment, the term “pharmaceutically acceptable carriers” includes, but is not limited to, may refer to 0.01-0.1M and preferably 0.05M phosphate buffer, or in another embodiment 0.8% saline. Additionally, such pharmaceutically acceptable carriers may be in another embodiment aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In one embodiment the level of phosphate buffer used as a pharmaceutically acceptable carrier is between about 0.01 to about 0.1M, or between about 0.01 to about 0.09M in another embodiment, or between about 0.01 to about 0.08M in another embodiment, or between about 0.01 to about 0.07M in another embodiment, or between about 0.01 to about 0.06M in another embodiment, or between about 0.01 to about 0.05M in another embodiment, or between about 0.01 to about 0.04M in another embodiment, or between about 0.01 to about 0.03M in another embodiment, or between about 0.01 to about 0.02M in another embodiment, or between about 0.01 to about 0.015 in another embodiment.

In one embodiment, the compounds of this invention may include compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

The pharmaceutical preparations comprising the compositions used in one embodiment in the methods provided herein, can be prepared by known dissolving, mixing, granulating, or tablet-forming processes. For oral administration, the active ingredients, or their physiologically tolerated derivatives in another embodiment, such as salts, esters, N-oxides, and the like are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. Examples of suitable inert vehicles are conventional tablet bases such as lactose, sucrose, or cornstarch in combination with binders such as acacia, cornstarch, gelatin, with disintegrating agents such as cornstarch, potato starch, alginic acid, or with a lubricant such as stearic acid or magnesium stearate.

Examples of suitable oily vehicles or solvents are vegetable or animal oils such as sunflower oil or fish-liver oil. Preparations can be effected both as dry and as wet granules. For parenteral administration (subcutaneous, intravenous, intraarterial, or intramuscular injection), the active ingredients or their physiologically tolerated derivatives such as salts, esters, N-oxides, and the like are converted into a solution, suspension, or emulsion, if desired with the substances customary and suitable for this purpose, for example, solubilizers or other auxiliaries. Examples are sterile liquids such as water and oils, with or without the addition of a surfactant and other pharmaceutically acceptable adjuvants. Illustrative oils are those of petroleum, animal, vegetable, or synthetic origin, for example, peanut oil, soybean oil, or mineral oil. In general, water, saline, aqueous dextrose and related sugar solutions, and glycols such as propylene glycols or polyethylene glycol are preferred liquid carriers, particularly for injectable solutions.

In addition, the composition described in the embodiments provided herein, can contain minor amounts of auxiliary substances such as wetting or emulsifying agents, pH buffering agents which enhance the effectiveness of the active ingredient.

An active component can be formulated into the composition as neutralized pharmaceutically acceptable salt forms. Pharmaceutically acceptable salts include the acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

In one embodiment, the compositions described herein, which are used in another embodiment, in the methods provided herein, further comprise a carrier, an excipient, a lubricant, a flow aid, a processing aid or a diluent.

The active agent is administered in another embodiment, in a therapeutically effective amount. The actual amount administered, and the rate and time-course of administration, will depend in one embodiment, on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington's Pharmaceutical Sciences.

Alternatively, targeting therapies may be used in another embodiment, to deliver the active agent more specifically to certain types of cell, by the use of targeting systems such as antibodies or cell specific ligands. Targeting may be desirable in one embodiment, for a variety of reasons, e.g. if the agent is unacceptably toxic, or if it would otherwise require too high a dosage, or if it would not otherwise be able to enter the target cells.

The compositions of the present invention are formulated in one embodiment for oral delivery, wherein the active compounds may be incorporated with excipients and used in the form of ingestible tablets, buccal tables, troches, capsules, elixirs, suspensions, syrups, wafers, and the like. The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. Syrup of elixir may contain the active compound sucrose as a sweetening agent methyl and propylparabens as preservatives, a dye and flavoring, such as cherry or orange flavor. In addition, the active compounds may be incorporated into sustained-release, pulsed release, controlled release or postponed release preparations and formulations.

Controlled or sustained release compositions include formulation in lipophilic depots (e.g. fatty acids, waxes, oils). Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors.

In one embodiment, the composition can be delivered in a controlled release system. For example, the agent may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In another embodiment, a controlled release system can be placed in proximity to the therapeutic target, i.e., the brain, thus requiring only a fraction of the systemic dose (see, e.g., Goodson, in Medical Applications of Controlled Release, supra, vol. 2, pp. 115-138 (1984). Other controlled release systems are discussed in the review by Langer (Science 249:1527-1533 (1990).

Such compositions are in one embodiment liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polglycolic acid, hydrogels, etc., or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils). Also comprehended by the invention are particulate compositions coated with polymers (e.g., poloxamers or poloxamines). Other embodiments of the compositions of the invention incorporate particulate forms, protective coatings, protease inhibitors, or permeation enhancers for various routes of administration, including parenteral, pulmonary, nasal, and oral.

In another embodiment, the compositions of this invention comprise one or more, pharmaceutically acceptable carrier materials.

In one embodiment, the carriers for use within such compositions are biocompatible, and in another embodiment, biodegradable. In other embodiments, the formulation may provide a relatively constant level of release of one active component. In other embodiments, however, a more rapid rate of release immediately upon administration may be desired. In other embodiments, release of active compounds may be event-triggered. The events triggering the release of the active compounds may be the same in one embodiment, or different in another embodiment. Events triggering the release of the active components may be exposure to moisture in one embodiment, lower pH in another embodiment, or temperature threshold in another embodiment. The formulation of such compositions is well within the level of ordinary skill in the art using known techniques. Illustrative carriers useful in this regard include microparticles of poly(lactide-co-glycolide), polyacrylate, latex, starch, cellulose, dextran and the like. Other illustrative postponed-release carriers include supramolecular biovectors, which comprise a non-liquid hydrophilic core (e.g., a cross-linked polysaccharide or oligosaccharide) and, optionally, an external layer comprising an amphiphilic compound, such as phospholipids. The amount of active compound contained in one embodiment, within a sustained release formulation depends upon the site of administration, the rate and expected duration of release and the nature of the condition to be treated suppressed or inhibited.

In one embodiment, the compositions of the invention are administered in conjunction with one or more therapeutic agents. These agents are in other embodiments, age spots removing agents, keratoses removing agents, analgesics, anesthetics, antiacne agents, antibacterial agents, antiyeast agents, antifungal agents, antiviral agents, antiburn agents, antidandruff agents, antidermatitis agents, antipruritic agents antiperspirants, antiinflammatory agents, antihyperkeratolytic agents, antidryskin agents, antipsoriatic agents, antiseborrheic agents, astringents, softeners, emollient agents, coal tar, bath oils, sulfur, rinse conditioners, foot care agents, hair growth agents, powder, shampoos, skin bleaches, skin protectants, soaps, cleansers, antiaging agents, sunscreen agents, wart removers, vitamins, tanning agents, topical antihistamines, hormones, vasodilators and retinoids.

In one embodiment, the compositions described herein, are used in the methods described herein. Accordingly and in another embodiment, provided herein is a method of treating a cardiovascular condition in a diabetic subject, comprising: contacting said subject with an effective amount of a composition comprising glutathione peroxidase or its isomer, metabolite, and/or salt therefore.

In one embodiment, the term “administering” refers to bringing a subject in contact with the compositions provided herein. For example, in one embodiment, the compositions provided herein are suitable for oral administration, whereby bringing the subject in contact with the composition comprises ingesting the compositions. A person skilled in the art would readily recognize that the methods of bringing the subject in contact with the compositions provided herein, will depend on many variables such as, without any intention to limit the modes of administration; the hemorrhagic event treated, age, pre-existing conditions, other agents administered to the subject, the severity of symptoms, location of the affected are and the like. In one embodiment, provided herein are embodiments of methods for administering the compounds of the present invention to a subject, through any appropriate route, as will be appreciated by one skilled in the art

In one embodiment, the methods provided herein, using the compositions provided herein, further comprise contacting the subject with one or more additional agent, which is not glutathione peroxidase or its isomer, metabolite, and/or salt therefore, nor vitamin-E. In one embodiment, the one or more additional agent not glutathione peroxidase or its isomer, metabolite, and/or salt therefore, nor vitamin-E, is an aldosterone inhibitor. In another embodiment, the additional agent is an angiotensin-converting enzyme. In another embodiment, the additional agent is an antioxidant. In another embodiment, the additional agent is an angiotensin receptor AT₁ blocker (ARB). In another embodiment, the additional agent is an angiotensin II receptor antagonist. In another embodiment, the additional agent is a calcium channel blocker. In another embodiment, the additional agent is a diuretic. In another embodiment, the additional agent is digitalis. In another embodiment, the additional agent is a beta blocker. In another embodiment, the additional agent is a statin. In another embodiment, the additional agent is a cholestyramine or in another embodiment, the additional agent is a combination thereof.

In one embodiment, the additional therapeutic agent used in the methods and compositions described herein is a statin. In another embodiment, the term “statins” refers to a family of compounds that are inhibitors of 3-hydroxy-3-methylglutaryl coenzyme A (HMG-CoA) reductase, the rate-limiting enzyme in cholesterol biosynthesis. As HMG-CoA reductase inhibitors, in one embodiment, statins reduce plasma cholesterol levels in various mammalian species.

Statins inhibit in one embodiment, cholesterol biosynthesis in humans by competitively inhibiting the 3-hydroxy-3-methyl-glutaryl-coenzyme A (“HMG-CoA”) reductase enzyme. HMG-CoA reductase catalyzes in another embodiment, the conversion of HMG to mevalonate, which is the rate determining step in the biosynthesis of cholesterol. Decreased production of cholesterol causes in one embodiment, an increase in the number of LDL receptors and corresponding reduction in the concentration of LDL particles in the bloodstream. Reduction in the LDL level in the bloodstream reduces the risk of coronary artery disease.

Statins used in the compositions and methods of the invention are lovastatin (referred to as mevinolin in one embodiment, or monacolin-K in another embodiment), compactin (referred to as mevastatin in one embodiment, or ML-236B in another embodiment), pravastatin, atorvastatin (Lipitor) rosuvastatin (Crestor) fluvastatin (Lescol), simvastatin (Zocor), cerivastatin. In one embodiment, the statin used as one or more additional therapeutic agent, is any one of the statins described herein, or in another embodiment, in combination of statins. A person skilled in the art would readily recognize that the choice of statin used, will depend on several factors, such as in certain embodiment, the underlying condition of the subject, other drugs administered, other pathologies and the like.

In one embodiment, the additional agent may be an anti-dyslipidemic agent such as (i) bile acid sequestrants such as, cholestyramine, colesevelem, colestipol, dialkylaminoalkyl derivatives. of a cross-linked dextran; Colestid™; LoCholest™; and Questran™, and the like; (ii) HMG-CoA reductase inhibitors such as atorvastatin, itavastatin, fluvastatin, lovastatin, pravastatin, rivastatin, rosuvastatin, simvastatin, and ZD-4522, and the like; (iii) HMG-CoA synthase inhibitors; (iv) cholesterol absorption inhibitors such as stanol esters, beta-sitosterol, sterol glycosides such as tiqueside; and azetidinones such as ezetimibe, vytorin, and the like; (v) acyl coenzyme A-cholesterol acyl transferase (ACAT) inhibitors such as avasimibe, eflucimibe, KY505, SMP 797, and the like; (vi) CETP inhibitors such as JTT 705,torcetrapib, CP 532,632, BAY63-2149, SC 591, SC 795, and the like; (vii) squalene synthetase inhibitors; (viii) anti-oxidants such as probucol, and the like; (ix) PPAR.alpha. agonists such as beclofibrate, benzafibrate, ciprofibrate, clofibrate, etofibrate, fenofibrate, gemcabene, and gemfibrozil, GW 7647, BM 170744, LY518674; and other fibric acid derivatives, such as Atromid™, Lopid™ and Tricor™, and the like; (x) FXR receptor modulators such as GW 4064, SR 103912, and the like; (xi) LXR receptor such as GW 3965, T9013137, and XTCO179628, and the like; (xii) lipoprotein synthesis inhibitors such as niacin; (xiii) rennin angiotensin system inhibitors; (xiv) PPAR or partial agonists; (xv) bile acid reabsorption inhibitors, such as BARI 1453, SC435, PHA384640, S892.1, AZD7706, and the like; (xvi) PPAR.delta. agonists such as GW 501516, and GW 590735, and the like; (xvii) triglyceride synthesis inhibitors; (xviii) microsomal triglyceride transport (MTTP) inhibitors, such as inplitapide, LAB687, and CP346086, and the like; (xix) transcription modulators; (xx) squalene epoxidase inhibitors; (xxi) low density lipoprotein (LDL) receptor inducers; (xxii) platelet aggregation inhibitors; (xxiii) 5-LO or FLAP inhibitors; and (xiv) niacin receptor agonists.

In another embodiment, the additional agent administered as part of the compositions, used in the methods provided herein, is an anti-hypertensive agents such as (i) diuretics, such as thiazides, including chlorthalidone, chlorothiazide, dichlorphenamide, hydroflumethiazide, indapamide, and hydrochlorothiazide; loop diuretics, such as bumetamide, ethacrynic acid, furosemide, and torsemide; potassium sparing agents, such as amiloride, and triamteren; and aldosterone antagonists, such as spironolactone, epirenone, and the like; (ii) beta-adrenergic blockers such as acebutolol, atenolol, betaxolol, bevantolol, bisoprolol, bopindolol, carteolol, carvedilol, celiprolol, esmolol, indenolol, metaprolol, nadolol, nebivolol, penbutolol, pindolol, propranolol, sotalol, tertatolol, tilisolol, and timolol, and the like; (iii) calcium channel blockers such as amlodipine, aranidipine, azelnidipine, barnidipine, benidipine, bepridil, cinaldipine, clevidipine, diltiazem, efonidipine, felodipine, gallopamil, isradipine, lacidipine, lemildipine, lercanidipine, nicardipine, nifedipine, nilvadipine, nimodepine, nisoldipine, nitrendipine, manidipine, pranidipine, and verapamil, and the like; (iv) angiotensin converting enzyme (ACE) inhibitors such as benazepril; captopril; cilazapril; delapril; enalapril; fosinopril; imidapril; losinopril; moexipril; quinapril; quinaprilat; ramipril; perindopril; perindropril; quanipril; spirapril; tenocapril; trandolapril, and zofenopril, and the like; (v) neutral endopeptidase inhibitors such as omapatrilat, cadoxatril and ecadotril, fosidotril, sampatrilat, AVE7688, ER4030, and the like; (vi) endothelin antagonists such as tezosentan, A308165, and YM62899, and the like; (vii) vasodilators such as hydralazine, clonidine, minoxidil, and nicotinyl alcohol, and the like; (viii) angiotensin II receptor antagonists such as candesartan, eprosartan, irbesartan, losartan, pratosartan, tasosartan, telmisartan, valsartan, and EXP-3137, FI6828K, and RNH6270, and the like; (ix) α/β adrenergic blockers as nipradilol, arotinolol and amosulalol, and the like; (x) alpha 1 blockers, such as terazosin, urapidil, prazosin, bunazosin, trimazosin, doxazosin, naffopidil, indoramin, WHIP 164, and XEN010, and the like; and (xi)-alpha 2 agonists such as lofexidine, tiamenidine, moxonidine, rilmenidine and guanobenz, and the like. Combinations of anti-obesity agents and diuretics or beta blockers may further include vasodilators, which widen blood vessels. Representative vasodilators useful in the compositions and methods of the present invention include, but are not limited to, hydralazine (apresoline), clonidine (catapres), minoxidil (loniten), and nicotinyl alcohol (roniacol).

The renin-angiotensin-aldosterone system (“RAAS”) is involved in one embodiment, in regulating pressure homeostasis and also in the development of hypertension, a condition shown as a major factor in the progression of cardiovascular diseases. Secretion of the enzyme renin from the juxtaglomerular cells in the kidney activates in another embodiment, the renin-angiotensin-aldosterone system (RAAS), acting on a naturally-occurring substrate, angiotensinogen, to release in another embodiment, a decapeptide, Angiotensin I. Angiotensin converting enzyme (“ACE”) cleaves in one embodiment, the secreated decapeptide, producing an octapeptide, Angiotensin II, which is in another embodiment, the primary active species of the RAAS system. Angiotensin II stimulates in one embodiment, aldosterone secretion, promoting sodium and fluid retention, inhibiting renin secretion, increasing sympathetic nervous system activity, stimulating vasopressin secretion, causing a positive cardiac inotropic effect or modulating other hormonal systems in other embodiments.

In one embodiment, the angiotensin converting enzyme (ACE) inhibitor used in the methods and compositions of the invention is captopril, cilazapril, delapril, enalapril, fentiapril, fosinopril, indolapril, lisinopril, perindopril, pivopril, quinapril, ramipril, spirapril, trandolapril, zofenopril or a combination thereof.

A representative group of ACE inhibitors consists in another embodiment, of the following compounds: AB-103, ancovenin, benazeprilat, BRL-36378, BW-A575C, CGS-13928C, CL-242817, CV-5975, Equaten, EU-4865, EU-4867, EU-5476, foroxymithine, FPL 66564, FR-900456, Hoe-065, I5B2, indolapril, ketomethylureas, KRI-1177, KRI-1230, L-681176, libenzapril, MCD, MDL-27088, MDL-27467A, moveltipril, MS-41, nicotianamine, pentopril, phenacein, pivopril, rentiapril, RG-5975, RG-6134, RG-6207, RGH-0399, ROO-911, RS-10085-197, RS-2039, RS 5139, RS 86127, RU-44403, S-8308, SA-291, spiraprilat, SQ-26900, SQ-28084, SQ-28370, SQ-23940, SQ-31440, Synecor, utibapril, WF-10129, Wy-44221, Wy-44655, Y-23785, Yissum P-0154, zabicipril, Asahi Brewery AB-47, alatriopril, BMS 182657, Asahi Chemical C-ill, Asahi Chemical C-112, Dainippon DU-1777, mixanpril, Prentyl, zofenoprilat, 1-(-(1-carboxy-6-(4-piperidinyl)hexyl)amino)-1-oxopropyl octahydro-1H-indole-2-carboxylic acid, Bioproject BP1.137, Chiesi CHF 1514, Fisons FPL-6564, idrapril, Marion Merrell Dow MDL-100240, perindoprilat and Servier S-5590, alacepril, benazepril, captopril, cilazapril, delapril, enalapril, enalaprilat, fosinopril, fosinoprilat, imidapril, lisinopril, perindopril, quinapril, ramipril, saralasin acetate, temocapril, trandolapril, ceranapril, moexipril, quinaprilat and spirapril.

In one embodiment, the terms “aldosterone antagonist” and “aldosterone receptor antagonist” refer to a compound that inhibits the binding of aldosterone to mineralocorticoid receptors, thereby blocking the biological effects of aldosterone. In one embodiment, the term “antagonist” in the context of describing compounds according to the invention refers to a compound that directly or in another embodiment, indirectly inhibits, or in another embodiment suppresses Aldosterone activity, function, ligand mediated transcriptional activation, or in another embodiment, signal transduction through the receptor. In one embodiment, antagonists include partial antagonists and in another embodiment full antagonists. In one embodiment, the term “full antagonist” refers to a compound that evokes the maximal inhibitory response from the Aldosterone, even when there are spare (unbound) Aldosterone present. In another embodiment, the term “partial antagonist” refers to a compound does not evoke the maximal inhibitory response from the androgen receptor, even when present at concentrations sufficient to saturate the androgen receptors present.

The aldosterone antagonists used in the methods and compositions of the present invention are in one embodiment, spirolactone-type steroidal compounds. In another embodiment, the term “spirolactone-type” refers to a structure comprising a lactone moiety attached to a steroid nucleus, such as, in one embodiment, at the steroid “D” ring, through a spiro bond configuration. A subclass of spirolactone-type aldosterone antagonist compounds consists in another embodiment, of epoxy-steroidal aldosterone antagonist compounds such as eplerenone. In one embodiment, spirolactone-type antagonist compounds consists of non-epoxy-steroidal aldosterone antagonist compounds such as spironolactone. In one embodiment, the invention provides a composition comprising an aldosterone antagonist, its isomer, functional derivative, synthetic analog, pharmaceutically acceptable salt or combination thereof; and a glutathione peroxidase or its isomer, functional derivative, synthetic analog, pharmaceutically acceptable salt or combination thereof, wherein the aldosterone antagonist is epoxymexrenone, or eplerenone, dihydrospirorenone, 2,2; 6,6-diethlylene-3oxo-17alpha-pregn-4-ene-21,17-carbolactone, spironolactone, 18-deoxy aldosterone, 1,2-dehydro-18-deoxyaldosterone, RU28318 or a combination thereof in other embodiments.

In one embodiment, the antioxidants include small-molecule antioxidants and antioxidant enzymes. Suitable small-molecule antioxidants include, in another embodiment, hydralazine compounds, glutathione, vitamin C, vitamin E, cysteine, N-acetyl-cysteine, .beta.-carotene, ubiquinone, ubiquinol-10, tocopherols, coenzyme Q, and the like. Suitable antioxidant enzymes include in one embodiment superoxide dismutase, catalase, glutathione peroxidase, or a combination thereof. Suitable antioxidants are described more fully in the literature, such as in Goodman and Gilman, The Pharmacological Basis of Therapeutics (9th Edition), McGraw-Hill, 1995; and the Merck Index on CD-ROM, Twelfth Edition, Version 12:1, 1996.

In addition to a direct action on arteries and arterioles, angiotensin II (AII), is one of the most potent endogenous vasoconstrictors known, exerts in one embodiment, stimulation on the release of aldosterone from the adrenal cortex. Therefore, the renin-angiotensin system, (RAAS) by virtue of its participation in the control of renal sodium handling, plays an important role in cardiovascular hemeostasis.

In another embodiment, the angiotensin II receptor antagonist used in the compositions and methods of the invention is losartan, irbesartan, eprosartan, candesartan, valsartan, telmisartan, zolasartin, tasosartan or a combination thereof. Examples of angiotensin II receptor antagonists used in the compositions and methods of the invention are in one embodiment biphenyltetrazole compounds or biphenylcarboxylic acid compounds or CS-866, losartan, candesartan, valsartan or irbesartan in other embodiments. In one embodiment, where the above-mentioned compounds have asymmetric carbons, the angiotensin II receptor antagonists of the compositions and methods used in the present invention are optical isomers and mixtures of said isomers. In one embodiment, hydrates of the above-mentioned compounds are also included.

In one embodiment, Cyclic fluxes of Ca²⁺ between three compartments—cytoplasm, sarcoplasmic reticulum (SR), and sarcomere—account for excitation-contraction coupling. Depolarization triggers in another embodiment, entry of small amounts of Ca²⁺ through the L-type Ca²⁺ channels located on the cell membrane, which in one embodiment, prompts SR Ca²⁺ release by cardiac ryanodine receptors (RyR's), a process termed calcium-induced Ca²⁺ release. A rapid rise in cytosolic levels results in one embodiment, fostering Ca 2+-troponin-C interactions and triggering sarcomere contraction. In another embodiment, activation of the ATP-dependent calcium pump (SERCA) recycles cytosolic Ca²⁺ into the SR to restore sarcomere relaxation. In another embodiment, Ca²⁺ channel blockers inhibits the triggering of sarcomer contraction and modulate increase in cystolic pressure.

In one embodiment, calcium channel blockers, are amlodipine, aranidipine, barnidipine, benidipine, cilnidipine, clentiazem, diltiazen, efonidipine, fantofarone, felodipine, isradipine, lacidipine, lercanidipine, manidipine, mibefradil, nicardipine, nifedipine, nilvadipine, nisoldipine, nitrendipine, semotiadil, veraparmil, and the like. Suitable calcium channel blockers are described more fully in the literature, such as in Goodman and Gilman, The Pharmacological Basis of Therapeutics (9th Edition), McGraw-Hill, 1995; and the Merck Index on CD-ROM, Twelfth Edition, Version 12:1, 1996; and on STN Express, file phar and file registry, which can be used in the compositions and methods of the invention.

In another embodiment, the β-blocker used in the compositions and methods of the invention is propanalol, terbutalol, labetalol propranolol, acebutolol, atenolol, nadolol, bisoprolol, metoprolol, pindolol, oxprenolol, betaxolol or a combination thereof.

In one embodiment, angiotensin II receptor blocker (ARB) are used in the compositions and methods of the invention. Angiotensin II receptor blocker (ARB) refers in one embodiment to a pharmaceutical agent that selectively blocks the binding of AII to the AT₁ receptor. ARBs provide in another embodiment, a more complete blockade of the RAAS by preventing the binding of AII to its primary biological receptor (AII type 1 receptor [AT₁]).

In another embodiment, the ARB used in the methods and compositions of the invention is candesartan, eprosartan, irbesartan losartan, olmesartan, telmisartan, valsartan or a combination thereof.

In one embodiment, a diuretic is used in the methods and compositions of the invention. In another embodiment, the diuretic is chlorothiazide, hydrochlorothiazide, methylclothiazide, chlorothalidon, or a combination thereof.

In one embodiment, the additional agent used in the compositions provided herein is a non-steroidal anti-inflammatory drug (NSAID). In another embodiment, the NSAID is sodium cromoglycate, nedocromil sodium, PDE4 inhibitors, leukotriene antagonists, iNOS inhibitors, tryptase and elastase inhibitors, beta-2 integrin antagonists and adenosine 2a agonists. In one embodiment, the NSAID is ibuprofen; flurbiprofen, salicylic acid, aspirin, methyl salicylate, diflunisal, salsalate, olsalazine, sulfasalazine, indomethacin, sulindac, etodolac, tolmetin, ketorolac, diclofenac, naproxen, fenoprofen, ketoprofen, oxaprozin, piroxicam, celecoxib, and rofecoxiband a pharmaceutically acceptable salt thereof. In one embodiment, the NSAID component inhibits the cyclo-oxygenase enzyme, which has two (2) isoforms, referred to as COX-1 and COX-2. Both types of NSAID components, that is both non-selective COX inhibitors and selective COX-2 inhibitors are useful in accordance with the present invention.

In one embodiment, the compositions comprising an additional agent that is not glutathione peroxidase or its isomer, metabolite, and/or salt therefore, nor vitamin-E, are used in the therapies effected by the methods provided herein. Accordingly and in one embodiment, provided herein is a method of treating, or inhibiting or suppressing or reducing the symptoms of cardiovascular complication in a diabetic subject, comprising the step of obtaining a biological sample from the subject, determining the haptoglobin genotype expressed by the subject, wherein if the subject expresses Hp-2-2 allele, the subject will benefit from a high-dose supplementation of vitamin-E, as well as contacting the subject with a therapeutically effective amount of a GPx mimetic, represented by any one of the compounds of formula I-X or their combination, whereby the cardiovascular complication is cardiovascular death, myocardial infarct, stroke or their combination.

Reactive oxygen species and inflammation play critical roles in the myocardial injury associated with ischemia-reperfusion. In the cellular environment of Diabetes Melitus (DM), these processes appear to be markedly exacerbated due to the increased oxidative stress and inflammatory cytokine production associated with the hyperglycemic state. Accordingly, genetic differences in protection from oxidative stress and inflammation are expected to be important in determining infarct size after ischemia-reperfusion injury

In one embodiment, the term “treatment” refers to any process, action, application, therapy, or the like, wherein a subject, including a human being, is subjected to medical aid with the object of improving the subject's condition, directly or indirectly. In another embodiment, the term “treating” refers to reducing incidence, or alleviating symptoms, eliminating recurrence, preventing recurrence, preventing incidence, improving symptoms, improving prognosis or combination thereof in other embodiments.

“Treating” embraces in another embodiment, the amelioration of an existing condition. The skilled artisan would understand that treatment does not necessarily result in the complete absence or removal of symptoms. Treatment also embraces palliative effects: that is, those that reduce the likelihood of a subsequent medical condition. The alleviation of a condition that results in a more serious condition is encompassed by this term.

The term “preventing” refers in another embodiment, to preventing the onset of clinically evident pathologies associated with vascular complications altogether, or preventing the onset of a preclinically evident stage of pathologies associated with vascular complications in individuals at risk, which in one embodiment are subjects exhibiting the Hp-2 allele. In another embodiment, the determination of whether the subject carries the Hp-2 allele, or in one embodiment, which Hp allele, precedes the methods and administration of the compositions of the invention.

Cardiovascular disease (CVD) is the most frequent, severe and costly complication of type 2 diabetes. It is the leading cause of death among patients with type 2 diabetes regardless of diabetes duration. In one embodiment, allelic polymorphism contributes to the phenotypic expression of CVD in diabetic subjects. In another embodiment, the methods and compositions of the invention are used in the treatment of CVD in diabetic subjects.

The term “myocardial infarct” or “MI” refers in another embodiment, to any amount of myocardial necrosis caused by ischemia. In one embodiment, an individual who was formerly diagnosed as having severe, stable or unstable angina pectoris can be diagnosed as having had a small MI. In another embodiment, the term “myocardial infarct” refers to the death of a certain segment of the heart muscle (myocardium), which in one embodiment, is the result of a focal complete blockage in one of the main coronary arteries or a branch thereof. In one embodiment, subjects which were formerly diagnosed as having severe, stable or unstable angina pectoris, are treated according to the methods or in another embodiment with the compositions of the invention, upon determining these subjects carry the Hp-2 allele and are diabetic.

The term “ischemia-reperfusion injury” refers in one embodiment to a list of events including: reperfusion arrhythmias, microvascular damage, reversible myocardial mechanical dysfunction, and cell death (due to apoptosis or necrosis). These events may occur in another embodiment, together or separately. Oxidative stress, intracellular calcium overload, neutrophil activation, and excessive intracellular osmotic load explain in one embodiment, the pathogenesis and the functional consequences of the inflammatory injury in the ischemic-reperfused myocardium. In another embodiment, a close relationship exists between reactive oxygen species and the mucosal inflammatory process.

In another embodiment, the route of administration in the step of contacting in the methods of the invention, using the compositions described herein, is optimized for particular treatments regimens. If chronic treatment of cardiovascular complications is required, in one embodiment, administration will be via continuous subcutaneous infusion, using in another embodiment, an external infusion pump. In another embodiment, if acute treatment of vascular complications is required, such as in one embodiment, in the case of miocardial infarct, then intravenous infusion is used.

In one embodiment, the compositions provided herein are administered in conjunction with other therapeutical agents. Representative agents that can be used in combination with the compositions of the invention are agents used to treat diabetes such as insulin and insulin analogs (e.g. LysPro insulin); GLP-1 (7-37) (insulinotropin) and GLP-1 (7-36)—NH.sub.2; biguanides: metformin, phenformin, buformin; .alpha.2-antagonists and imidazolines: midaglizole, isaglidole, deriglidole, idazoxan, efaroxan, fluparoxan; sulfonylureas and analogs: chlorpropamide, glibenclamide, tolbutamide, tolazamide, acetohexamide, glipizide, glimepiride, repaglinide, meglitinide; other insulin secretagogues: linogliride, A-4166; glitazones: ciglitazone, pioglitazone, englitazone, troglitazone, darglitazone, rosiglitazone; PPAR-gamma agonists; fatty acid oxidation inhibitors: clomoxir, etomoxir; .alpha.-glucosidase inhibitors: acarbose, miglitol, emiglitate, voglibose, MDL-25,637, camiglibose, MDL-73,945; beta.-agonists: BRL 35135, BRL 37344, Ro 16-8714, ICI D7114, CL 316,243; phosphodiesterase inhibitors: L-386,398; lipid-lowering agents: benfluorex; antiobesity agents: fenfluramine; vanadate and vanadium complexes (e.g. Naglivan®)) and peroxovanadium complexes; amylin antagonists; glucagon antagonists; gluconeogenesis inhibitors; somatostatin analogs and antagonists; antilipolytic agents: nicotinic acid, acipimox, WAG 994. Also contemplated for use in combination with the compositions of the invention are pramlintide acetate (Symlin®), AC2993, glycogen phosphorylase inhibitor and nateglinide. Any combination of agents can be administered as described hereinabove.

The term “subject” refers in one embodiment to a mammal including a human in need of therapy for, or susceptible to, a condition or its sequelae. The subject may include dogs, cats, pigs, cows, sheep, goats, horses, rats, and mice and humans. The term “subject” does not exclude an individual that is normal in all respects.

The following examples are presented in order to more fully illustrate the preferred embodiments of the invention. They should in no way be construed, however, as limiting the broad scope of the invention.

### EXAMPLES

### Materials and Methods

Participants

Study Location

The study protocol was approved by the Independent Ethics Committee (IEC) of the Carmel Medical Center in Clalit Health Services (CHS) and the Israeli Ministry of Health. The study took place within 47 primary health care clinics in the Haifa and Western Galilee district of CHS. Routine care and follow up of all DM patients in these clinics is provided by the patient's family primary care physician and a designated DM nurse.

Eligibility

Patients were eligible for inclusion in the study if they had Type II DM and were 55 years of age or older. 22,142 individuals were identified meeting these requirements in the 47 health clinics described above. Study exclusion criterion were (1) uncontrolled hypertension; (2) myocardial infarction or stroke within 1 month prior to enrollment; (3) unwillingness to stop antioxidant supplements; (4) known allergy to vitamin E.

Potentially eligible patients were invited by their primary care physician to undergo Hp typing between April 2005 and April 2006. Discretion was left to the primary care physician to only invite those patients believed to be able to comply with the study requirements. All patients undergoing Hp typing signed an informed consent form (ICF) explicitly stating that they had consented to Hp typing to identify their cardiovascular risk. These patients were also aware that their Hp type would determine if they were eligible to be enrolled in a study in which they would be randomized for treatment with either placebo or vitamin E. However, as stipulated by the IEC, patients were required to sign an additional ICF prior to receiving and beginning treatment with vitamin E or placebo. Patients understood that consent to undergo Hp typing in no way indicated an agreement to participate in the treatment phase of the study.

Hp phenotyping was performed in the Hp core laboratory on hemoglobin-enriched serum by polyacrylamide electrophoresis An Hp phenotype (Hp 1-1, Hp 2-1 or Hp 2-2) is obtained using this method in over 98% of individuals with a reproducibility of greater than 99%.²⁶ This method provides a signature banding pattern for each of the three possible Hp phenotypes with which we have demonstrated 100% correspondence to the three possible Hp genotypes of identical nomenclature as determined by PCR Individuals with Hp 2-2 were approached by their primary care physician and consented to participate in the treatment phase of the study. Only after signing this second ICF were patients provided with a medication bottle to begin the treatment phase of the study.

Interventions and Monitoring Compliance

DM individuals with the Hp 2-2 genotype providing consent for the treatment phase of the study were randomly allocated to receive either placebo or vitamin E (natural source d-alpha tocopherol) at a dose of 400 IU per day for the duration of the study. Placebo pills were identical to vitamin E pills except that they contained no vitamin E. Pills were supplied in bottles identical in appearance having only the participant's enrollment number on the bottle. Treatment allocation was blinded for all study participants, physicians and the study staff. All treatment decisions regarding routine care remained at the discretion of the patient's primary care physician. Assessment of compliance was based on telephone interviews.

Randomization Procedure

A computer generated randomization list was used to assign individuals to the two treatment groups. At the site of study drug manufacture all medication bottles were labeled with a number in accordance with the computer generated randomization key. A medication bottle number was assigned to potential participants in the study coordination center after receiving formal documentation from the Hp core laboratory that an individual was Hp 2-2. The coordination center then assigned that individual the next available bottle number in sequence and that bottle was sent to the patient's primary care clinic where it was to be distributed by the primary physician only after the individual consented to participate in the treatment phase of the study and signed the second ICF. A large number of Hp 2-2 patients who underwent randomization declined to sign the second ICF and therefore never received the study medication. This rather atypical study design was adopted due to the limited financial resources of this study with no dedicated study personnel in the clinics as well as due to the requirement by the IEC for a two phased consent for the study. Administratively, randomization could only be performed by the central facility and it was felt that if the patient was randomized only after signing the second ICF, necessitating yet a third visit to the clinic to finally receive the study medication, that the interval from patient recruitment to treatment would be dramatically lengthened and the size of the treatment cohort would be dramatically reduced. It is critical to note that the identity of the contents of the bottles was not known to any participant, physician, or individual involved in the study during enrollment, randomization, follow-up or adjudication of events. Critically, patients who were randomized but did not sign the second ICF and did not begin treatment, were unaware to what treatment group they had been assigned.

Primary and Secondary Outcomes

The primary outcome of the study was the composite of cardiovascular death, non-fatal myocardial infarction and stroke. Cardiovascular death was defined as either (1) unexplained death due to ischemic cardiovascular disease occurring within 24 hours after the onset of symptoms or (2) death from myocardial infarction or stroke within 7 days after the myocardial infarction or stroke. Myocardial infarction was defined by the typical rise and fall of serum markers of myocardial necrosis (CK-MB or troponin) with at least one of the following: (a) typical ischemic symptoms; (b) development of pathologic Q-waves on the ECG; (c) ECG changes diagnostic of ischemia.²⁷ Stroke was defined as a neurologic deficit lasting more than 24 hours. Prespecified secondary endpoints were: total mortality, hospitalization for congestive heart failure, and coronary revascularization.

Sample Size Determination

Sample size and power calculations were based on the incidence of primary events in HOPE in Hp 2-2 individuals who did and did not receive vitamin E It was calculated that 500 Hp 2-2 participants would be needed in each treatment group in order to achieve 80% power to detect a 45% reduction in the primary composite endpoint after four years of treatment at a significance level of p<0.05.

Ascertainment and Adjudication of Events

All CHS hospitalizations, as well as out of hospital deaths, are documented in a computerized database. Events were ascertained by reviewing all hospitalizations of study participants. Adjudication of events corresponding to the primary and secondary outcomes was based on the hospitalization discharge summary by a panel of physicians blinded to treatment allocation. For out-of-hospital deaths, adjudication was based on interviews with the patient's physician and family.

Interim Analysis of Data for Safety and Efficacy and Termination of the Study

The data were reviewed at one year following initiation of the study, and were to be reviewed every six months thereafter. As will be outlined in Results, the one year review led to early termination of the study.

Study Registry

All patients for whom an Hp type was obtained but who did not enroll in the treatment phase were enrolled in the registry. Follow up for the patients in the registry was done at the same time and using the same methodology for outcomes adjudication as for patients in the treatment study group. The registry included all Hp 1-1 and Hp 2-1 individuals who were Hp typed. Hp 2-2 individuals in the registry were those who chose not to participate in the treatment phase or for whom the randomization phase was closed. Registry patients were not treated by the study medication but were followed in order to assess the ability of the Hp type to prospectively determine cardiovascular risk.

Statistical Analysis

Hp 2-2 individuals who were assigned a medication bottle number by the study coordination center but refused to enter the treatment phase of the study were not included in the treatment group analysis and as they were never provided with or treated with the study medication. Rather, these non-treated Hp 2-2 individuals were analyzed as part of the registry analysis. Categorical data are presented as absolute values and percentages. Differences in demographic variables and medications between the two groups were compared by chi-squared test. Kaplan-Meier estimates, stratified according to the treatment or according to the Hp genotype for the primary composite endpoint, are presented as event curves. Statistical analysis was performed using SPSS statistical software Version 12.0. All reported p-values are two-sided.

### Example 1

### Eligibility, Recruitment and Allocation

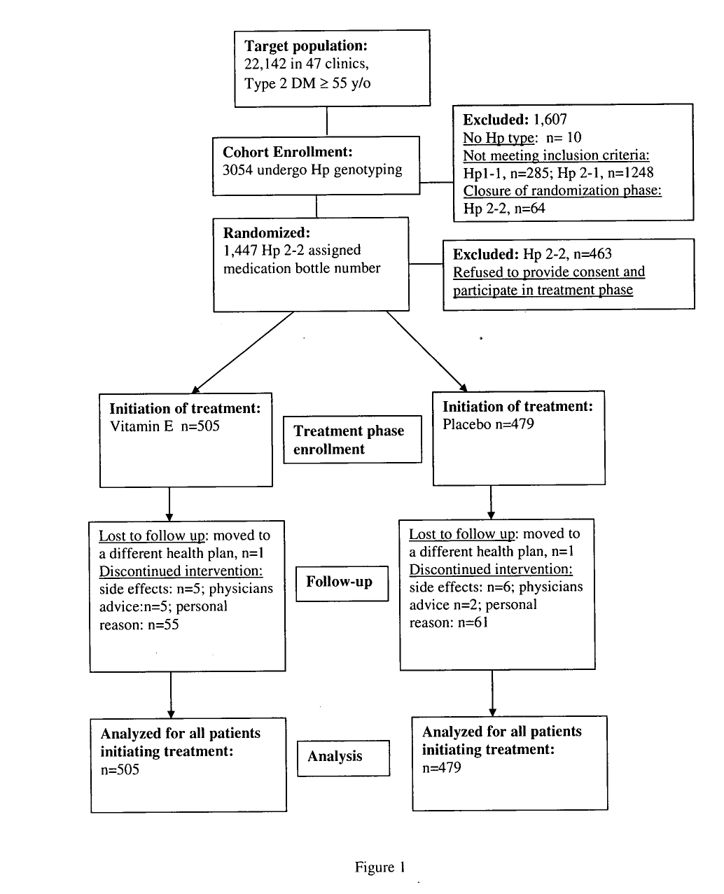

FIG. 1 provides a flow diagram of the trial comparing vitamin E versus placebo in individuals with the Hp 2-2 genotype and DM.

From a target population of 22,142 individuals, 3054 underwent Hp genotyping between April, 2005 and April, 2006. An Hp genotype was obtained on 3044 individuals with the distribution: Hp 1-1 285 (9.4%); Hp 2-1 1248 (41.0%); Hp 2-2 1511 (49.6%). Hp 1-1 and Hp 2-1 individuals were excluded from randomization but were followed in a registry for primary and secondary endpoints. Of 1511 DM individuals identified as Hp 2-2, 527 were excluded from the treatment phase of the study due to either closure of the randomization phase of the study or due to a refusal to sign consent to participate in the treatment phase of the study. These 527 DM individuals were also followed in the registry. As a result a total of 984 Hp 2-2 DM individuals were randomized and treated with vitamin E (505) or placebo (479).

### Example 2

### Baseline Demographic and Clinical Characteristics of Study Participants

The baseline characteristics of Hp 2-2 DM individuals receiving placebo or vitamin E are shown in Table 1. The prevalence of cardiovascular disease in this study cohort at baseline was 25%. The only significant difference between the groups was in statin use which was greater in the Hp 2-2 placebo group (p=0.02).

TABLE 1
Baseline characteristics of treatment groups

| | |
|---|---|
| Hp 2-2 Vit E | Hp 2-2 Placebo |

| | | |
|---|---|---|
| N | 505 | 479 |

Demographic data

| | | | | |
|---|---|---|---|---|
| Mean age (SD) years | 68.3 | (8.0) | 68.9 | (7.8) |
| Duration of DM (SD) | 10.5 | (8.4) | 11.2 | (7.9) |
| Males [n (%)] | 237 | (46.9) | 234 | (48.8) |
| Minorities [n (%)] | 80 | (15.8) | 78 | (16.3) |

History [n (%)]

| | | | | |
|---|---|---|---|---|
| Myocardial infarction | 68 | (13.4) | 67 | (14.0) |
| Stroke | 36 | (7.1) | 30 | (6.2) |
| PCI | 54 | (10.7) | 55 | (11.5) |
| CABG | 41 | (8.1) | 48 | (10.0) |
| Hypertension | 369 | (73.0) | 363 | (75.8) |
| Past smoker | 144 | (28.5) | 118 | (24.6) |
| Current smoker | 56 | (11.0) | 62 | (12.9) |

Lab Results (mean (SD))

| | | | | |
|---|---|---|---|---|
| HbA1c | 7.3 | (1.3) | 7.4 | (1.2) |
| Creatinine (umol/1) | 0.9 | (0.3) | 0.9 | (0.3) |
| Total cholesterol (mg/dl) | 187.5 | (33.2) | 187.1 | (33.8) |
| HDL (mg/dl) | 46.1 | (11.0) | 46.2 | (11.1) |
| LDL (mg/dl) | 104.2 | (25.6) | 102.1 | (26.5) |

Medications [n (%)]

| | | | | |
|---|---|---|---|---|
| Aspirin | 197 | (39.0) | 183 | (38.1) |
| Statins | 266 | (52.6) | 287 | (59.8)* |
| B-blockers | 196 | (38.8) | 187 | (38.9) |
| ACE inhibitors | 239 | (47.3) | 254 | (52.9) |
| Metformin | 323 | (64.0) | 284 | (59.2) |

*p = 0.02 increased statin use in placebo group. No other significant differences between groups in any other variable.

### Example 3

### Follow Up

Two participants were lost to follow up (one in each group). 7 individuals discontinued intervention due to advice from a physician (5 in vitamin E group, 2 in placebo). 11 individuals discontinued the study due to perceived side effects (5 in vitamin E and 6 in placebo). 55 participants taking vitamin E and 61 participants taking placebo were non-compliant with taking the respective pills based on telephone interviews.

### Example 4

### Outcome

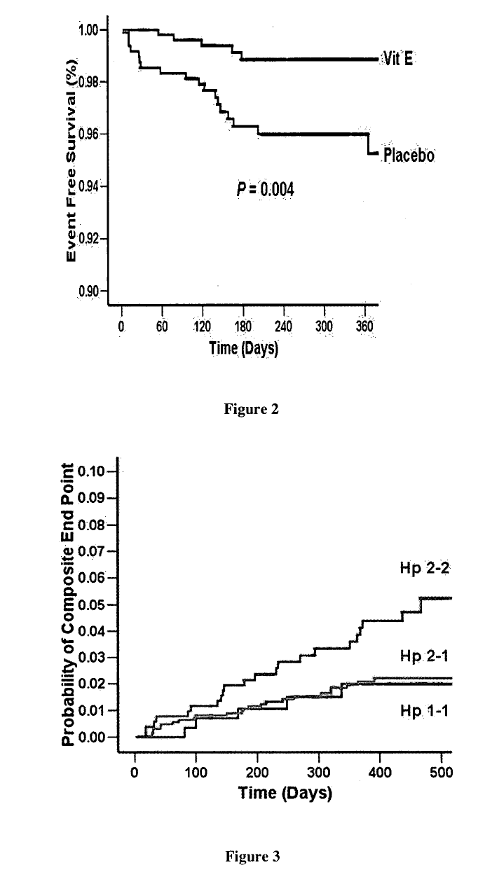

At the first interim analysis of study outcomes (one year after initiation of the study) the primary study outcome was significantly reduced in participants receiving vitamin E when compared to placebo (1.0% for vitamin E vs. 3.8% for placebo, Hazard Ratio (HR) 0.26, 95% CI 0.13-0.69, p=0.004). This finding reflected a stronger effect than was anticipated in the study design, and led to termination of the study, three years prior to the anticipated date. A Kaplan-Meier plot of the primary composite outcome comparing the vitamin E and placebo groups is shown in FIG. 2. The reduction in the primary outcome in the vitamin E group was predominately due to a significant reduction in the incidence of non-fatal myocardial infarction (Table 2). None of the prespecified secondary outcomes were significantly different between the two treatment groups (Table 2).

TABLE 2
Primary and secondary endpoint analysis of treatment outcomes

| Endpoint | Vitamin E | Placebo | p |
|---|---|---|---|
| Primary Composite, n (%) | 5 (1.0%) | 18 (3.8%) | 0.004 |
| Myocardial infarction, n (%) | 1 (0.2%) | 10 (2.1%) | 0.004 |
| Stroke, n (%) | 1 (0.2%) | 4 (0.8%) | 0.16 |
| Cardiovascular Death, n (%) | 3 (0.59%) | 4 (0.8%) | 0.65 |

Secondary Outcomes:

| | | | |
|---|---|---|---|
| Revascularization, n (%) | 5 (1.0%) | 7 (1.5%) | 0.50 |
| Congestive Heart Failure, n (%) | 4 (0.8%) | 2 (0.4%) | 0.45 |
| Total mortality, n (%) | 7 (1.4%) | 7 (1.5%) | 0.92 |

### Example 5

### Registry Outcomes

There was no difference in the baseline characteristics of patients in the registry with the Hp 1-1, Hp 2-1 and Hp 2-2 genotypes similar to what has been previously described in other cohorts. ²⁰⁻²³ However, the primary composite outcome was significantly increased in Hp 2-2 individuals in the registry as compared to non-Hp 2-2 individuals in the registry (4.2% versus 2.0%, p=0.005 by log-rank analysis as shown in FIG. 3). Furthermore, consistent with the main study outcome in individuals allocated to vitamin E or placebo, Hp 2-2 individuals randomized and treated with vitamin E had a significantly lower primary event rate compared to Hp 2-2 individuals in the registry (HR 0.34, 95% CI 0.18-0.87, p=0.02).

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

1. A method of determining prognosis for a diabetic subject having a cardiovascular complication, to benefit from supplementation of vitamin-E, comprising the step of obtaining a biological sample from the subject; and determining the subject's haptoglobin allelic genotype, whereby a subject expressing the Hp-2-2 genotype will benefit from supplementation of vitamin-E.

2. The method of claim 1, wherein said vascular complication is cardiovascular death, myocardial infarct, stroke or a combination thereof.

3. The method of claim 1, wherein said step of determining said haptoglobin genotype is effected by a method selected from a signal amplification method, a direct detection method, detection of at least one sequence change, immunological method or a combination thereof.

4.-9. (canceled)

10. The method of claim 1, whereby the prognosis comprises determining the importance of reducing oxidative stress.

11. A method of treating a diabetic subject having a cardiovascular complication, comprising the step of contacting the subject with an effective amount of a composition comprising glutathione peroxidase or its isomer, metabolite, and/or salt therefore, and vitamin-E, thereby treating vascular complication.

12. A method of inhibiting or suppressing a cardiovascular complication in a diabetic subject comprising the step of contacting the subject with an effective amount of a composition comprising glutathione peroxidase or its isomer, metabolite, and/or salt therefore, and vitamin-E, thereby inhibiting or suppressing vascular complication.

13. A method of reducing symptoms associated with a cardiovascular complication in a diabetic subject comprising the step of contacting the subject with an effective amount of a composition comprising glutathione peroxidase or its isomer, metabolite, and/or salt therefore, and vitamin-E, thereby inhibiting or suppressing vascular complication.

14. The method of claim 11, whereby said glutathione peroxidase, an isomer, a functional derivative, a synthetic analog, is represented by the compound of formula I:

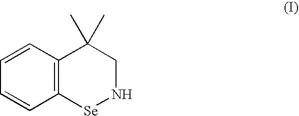

15. The method of claim 11, whereby said glutathione peroxidase or its isomer, metabolic, and/or salt therefore, is benzisoselen-azoline or -azine derivatives represented by the following general formula II: 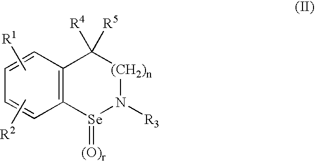
wherein R¹=R²=hydrogen; lower alkyl; OR⁶; —(CH₂)ₘNR⁶R⁷; —(CH₂)_{q}NH₂; —(CH₂)ₘNHSO₂(CH₂)₂NH₂; —NO₂; —CN; —SO₃H; —N⁺(R⁵)₂O⁻; F; Cl; Br; I; —(CH₂)ₘR⁸; —(CH₂)ₘCOR⁸; —S(O)NR⁶R⁷; —SO₂ NR⁶R⁷; —CO(CH₂)ₚCOR⁸; R⁹;
R³=hydrogen; lower allyl; aralkyl; substituted aralkyl; —(CH₂)ₘ COR⁸; —(CH₂)_{q}R⁸; —CO(CH₂)ₚCOR⁸; —(CH₂)ₘSO₂R⁸; —(CH₂)ₘS(O)R⁸;
R⁴=lower alkyl; aralkyl; substituted aralkyl; —(CH₂)ₚCOR⁸; —(CH₂)ₚR⁸; F;
R⁵=lower alkyl; aralkyl; substituted aralkyl;
R⁶=lower alkyl; aralkyl; substituted aralkyl; —(CH₂)ₘCOR⁸; —(CH₂)_{q}R⁸;
R⁷ lower alkyl; aralkyl; substituted aralkyl; —(CH₂)ₘCOR⁸;
R⁸-lower alkyl; aralkyl; substituted aralkyl; aryl; substituted aryl; heteroaryl; substituted heteroaryl; hydroxy; lower alkoxy;
R⁹ is represented by any structure of the following formulae: 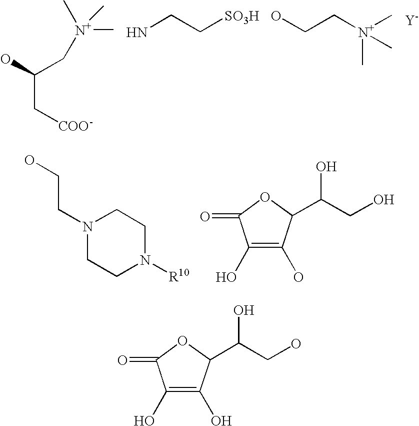
R¹⁰=hydrogen; lower alkyl; aralkyl or substituted aralkyl; aryl or substituted aryl;
Y⁻ represents the anion of a pharmaceutically acceptable acid;
n=0, 1; m=0, 1, 2; p=1, 2, 3; q=2, 3, 4; and
r=0, 1.

16. The method of claim 11, whereby the glutathione peroxidase or its isomer, metabolite, and/or salt therefore is represented by the compound of formula III: 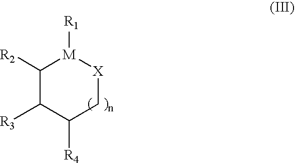
wherein,
the compound of formula 1 is a ring; and
X is O or NH
M is Se or Te
n is 0-2
R₁ is oxygen; and forms an oxo complex with M; or
R₁ is oxygen or NH; and
forms together with the metal, a 4-7 member ring, which optionally is substituted by an oxo or amino group; or
forms together with the metal, a first 4-7 member ring, which is optionally substituted by an oxo or amino group, wherein said first ring is fused with a second 4-7 member ring, wherein said second 4-7 member ring is optionally substituted by alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; and
R₂, R₃ and R₄ are independently hydrogen, alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; or R₂, R₃ or R₄ together with the organometallic ring to which two of the substituents are attached, form a fused 4-7 member ring system wherein said 4-7 member ring is optionally substituted by alkyl, alkoxy, nitro, aryl, cyano, hydroxy, amino, halogen, oxo, carboxy, thio, thioalkyl, or —NH(C═O)R^{A}, —C(—O)NR^{A}R^{B}, —NR^{A}R^{B} or —SO₂R where R^{A} and R^{B} are independently H, alkyl or aryl; wherein R₄ is not an alkyl; and
wherein if R₂, R₃ and R₄ are hydrogen and R₁ forms an oxo complex with M, n is 0 then M is Te; or
if R₂, R₃ and R₄ are hydrogen and R₁ is an oxygen that forms together with the metal an unsubstituted, saturated, 5 member ring, n is 0 then M is Te; or
if R₁ is an oxo group, and n is 0, R₂ and R₃ form together with the organometallic ring a fused benzene ring, R₄ is hydrogen, then M is Se; or
if R₄ is an oxo group, and R₂ and R₃ form together with the organometallic ring a fised benzene ring, R₁ is oxygen, n is 0 and forms together with the metal a first 5 member ring, substituted by an oxo group α to R₁, and said ring is fused to a second benzene ring, then M is Te.

17-22. (canceled)

23. The method of claim 11, whereby the glutathione peroxidase or its isomer, metabolite, and/or salt therefore is represented by the compound of formula IX: 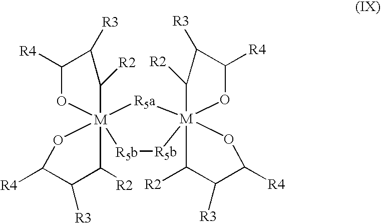
wherein,
M is Se or Te;
R₂, R₃ or R₄ are independently hydrogen, alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; or R₂, R₃ or R₄ together with the organometallic ring to which two of the substituents are attached, is a fused 4-7 member ring system, wherein said 4-7 member ring is optionally substituted by alkyl, alkoxy, nitro, aryl, cyano, hydroxy, amino, halogen, oxo, carboxy, thio, thioalkyl, or —NH(C═O)R^{A}, —C(═O)NR^{A}R^{B}, —NR^{A}R^{B} or —SO₂R where R^{A} and R^{B} are independently H, alkyl or aryl; and
R₅ₐ or R_{5b} is one or more oxygen, carbon, or nitrogen atoms and forms a neutral complex with the chalcogen.

24. The method of claim 23, whereby the compound of formula IX is represented by the compound of formula X:

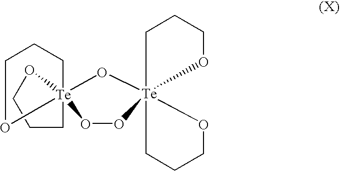

25. The method of claim 11, whereby the cardiovascular complication is myocardial infarct, cardiovascular death, stroke, or a combination thereof.

26. The method of claim 11, preceded by the step of determining the Hp phenotype in said subject.

27. The method of claim 11, whereby the step of contacting is via oral, intravenous, intraarterial, intramuscular subcutaneous, parenteral, transmucosal, transdermal, intracranial, or topical administration.

28. The method of claim 11, comprising contacting the subject with one or more additional agent, which is not glutathione peroxidase or its isomer, metabolite, and/or salt therefore, nor vitamin-E.

29. The method of claim 28, whereby the one or more additional agent not glutathione peroxidase or its isomer, metabolite, and/or salt therefore, nor vitamin-E, is an aldosterone inhibitor, and angiotensin-converting enzyme, an antioxidant, an angiotensin receptor AT₁ blocker (ARB), an angiotensin II receptor antagonist, a calcium channel blocker, a diuretic, digitalis, a beta blocker, a statin, a cholestyramine, a NSAID, or a combination thereof.

30. A composition for treating a cardiovascular complication in a subject comprising: a therapeutically effective amount of a composition comprising glutathione peroxidase or its isomer, metabolite, and/or salt therefore and vitamin-E.

31. The composition of claim 30, further comprising a carrier, an excipient, a lubricant, a flow aid, a processing aid, a diluent, or a combination thereof.

32. The composition of claim 31, wherein said carrier, excipient, lubricant, flow aid, processing aid or diluent is a gum, a starch, a sugar, a cellulosic material, an acrylate, calcium carbonate, magnesium oxide, talc, lactose monohydrate, magnesium stearate, colloidal silicone dioxide or mixtures thereof.

33. The composition of claim 30, wherein said glutathione peroxidase, its isomer, functional derivative, or synthetic analog and their combination is represented by the any one of the compounds of formula I-X, or their combination.

34. The composition of claim 30, comprising a binder, a disintegrant, a buffer, a protease inhibitor, a surfactant, a solubilizing agent, a plasticizer, an emulsifier, a stabilizing agent, a viscosity increasing agent, a sweetener, a film forming agent, or any combination thereof.

35. The composition of claim 30, wherein said composition is in the form of a pellet, a tablet, a capsule, a solution, a suspension, a dispersion, an emulsion, an elixir, a gel, an ointment, a cream, or a suppository.

36. The composition of claim 30, wherein said composition is in a form suitable for oral, intravenous, intraarterial, intramuscular, subcutaneous, parenteral, transmucosal, transdermal, intracranial, or topical administration.

37. The composition of claim 30, wherein said composition is a controlled release composition.

38. The composition of claim 30, wherein said composition is an immediate release composition.

39.-40. (canceled)

41. The composition of claim 30, wherein the cardiovascular complication is myocardial infarct, cardiovascular death, stroke, or a combination thereof.
